(19) United States
(12) Patent Application Publication
(10) Pub. No.: US 2007/0031396 A1
(43) Pub. Date: Feb. 8, 2007

(54) Method of treating neurological diseases

(75) Inventors: Malcom Maden, Teddington (GB); Jonathan Patrick Thomas Corcoran, London (GB)

Correspondence Address: MARSHALL, GERSTEIN & BORUN LLP, 233 S. WACKER DRIVE, SUITE 6300, SEARS TOWER, CHICAGO, IL, 60606, US

(73) Assignee: KINGS COLLEGE LONDON, London (GB)

(21) Appl. No.: 11/543,292

(22) Filed: Oct. 2, 2006

Related U.S. Application Data
(63) Continuation of application No. 10/468,244, filed on Jan. 20, 2004, now abandoned.

(30) Foreign Application Priority Data
Feb. 19, 2001 (GB) 0103998.1

(51) Int. Cl. A61K 38/44; A61K 48/00

(52) U.S. Cl. 424/94.4; 514/44

(57) ABSTRACT

The invention relates to a method of treating a condition in a subject comprising administering an effective amount of an agent to said subject wherein said agent modulates one or more components of the retinoid signaling pathway.

### FIELD OF THE INVENTION

The present invention relates to methods for treating a condition in a subject by modulation of one or more component(s) in the retinoid signalling pathway in said subject. The invention further relates to vectors comprising nucleic acids for use in said methods.

### BACKGROUND TO THE INVENTION

Vitamin A (retinol or all-trans retinol) and provitamin A (β-carotene) are metabolised to retinoid derivatives which function in light absorption for vision or gene regulation for growth and development (Duester 2000).

The metabolite required for vision is 11-cis-retinal which functions as a light-absorbing pigment in the retina (Wald, 1951). The metabolites all-trans-retinoic acid and 9-cis-retinoic acid act as ligands for the nuclear retinoid receptors that directly regulate gene expression. There are two classes of retinoid receptors, retinoic acid receptors (RARs) and retinoid X receptors (RXRs) (Kastner et al 1994; Nagpal and Chandraratna 1998). RARs are activated by all-trans-retinoic acid and 9-cis-retinoic acid and the RXRs are activated by only 9-cis-retinoic acid (Kastner et al 1994; Kliewer et al, 1994).

Retinoic acid has been shown to be important for birth survival and function of foetal neurones (Wuarin and Sidell, 1991; Quinn and DeBoni, 1991). Furthermore, studies on a variety of embryonic neuronal types have shown that retinoic acid can stimulate both the number and length of embryonic neurones (Maden, 1998; Corcoran and Maden, 1999, Corcoran et al, 2000). The requirement for RA in the developing CNS has been extensively studied (review Maden, 2001), but almost nothing is known about the nature of retinoid signalling, if any, in the adult CNS.

Amyotrophic lateral sclerosis (ALS) is a progressive, fatal, neurodegenerative disease characterised by the loss of motor neurones in the motor cortex, brain stem and spinal cord, this leads to weakness and atrophy (Delise and Carpenter, 1984; Mulder et al, 1986). ALS occurs in both sporadic (90% of all cases) and familial forms (10% of all cases) (Jackson and Bryan, 1998). In 20% of familial ALS, mutations have been found in the copper, zinc superoxide dismutase gene (SOD1) (Rosen, 1993; Deng et al, 1993). The genes involved in the sporadic cases and in the remaining 80% of familial cases have yet to be identified. Currently there is no treatment which prevents or reverses the course of the disorder. Available treatments (such as riluzole and antioxidants) can at best extend survival to a modest degree.

Parkinson's disease is a slowly progressive disorder that affects movement, muscle control, and balance. Parkinson's disease is not fatal, but it reduces longevity. It also seriously impairs the quality of life and may sometimes lead to severe incapacity within 10 to 20 years. The average age of onset is 55; about 10% of Parkinson's cases are in people younger than 40 years old. Parkinson's disease occurs when cells are destroyed in certain parts of the brain stem, particularly the crescent-shaped cell mass known as the substantia nigra. Nerve cells in the substantia nigra send out fibres to the corpus stratia. There the cells release dopamine, an essential neurotransmitter. Loss of dopamine in the corpus stratia is the primary defect in Parkinson's disease. This loss negatively effects the nerves and muscles controlling movement and co-ordination, resulting in the major symptoms characteristic of Parkinson's disease. Currently research is also being carried out into the role of the protein alpha synuclein, the complex I enzyme, NMDA receptors, immune factors, environmental factors (such as toxins, infections, industrial chemicals) and oxygen-free radicals in causing Parkinson's disease. The gene Parkin has been found to be responsible for a rare form of Parkinson's disease which occurs in children and adolescents.

The current drug treatments for Parkinson's disease are levodopa, anticholinergic drugs, amantadine, selegiline, dopamine agonists, catechol-o-methyl transferase inhibitors. These treatments are effective in alleviating symptoms and even slowing progression of the disease. However, over time, the side effects (neurological, such as dyskinesia, and psychiatric disturbances) of many of these medications can be nearly as distressing as the disease itself and the drugs may eventually lose their effectiveness. Drugs in development include those which block the action of glutamate (such as remacenide, dextromethrophan, riluzole and lamotrigine) and nicotinic acetylcholine receptor agonists.

Alzheimer's disease is a slow degenerative disease of the brain from which there is no recovery. The disease attacks nerve cells in all parts of the cortex thereby impairing a person's abilities to govern emotions, recognise errors and patterns, co-ordinate movement, and remember. Eventually, an afflicted person loses all memory and mental functioning. Research is being carried out into factors which play a role in Alzheimer's disease such as the tau protein in neurofibrillary tangles; β amyloid protein, amyloid precursor protein, endoplasmic-reticulum associated binding protein, AMY117 plaques, prostate apoptosis response-4, neurotransmnittors (such as acetylcholin, serotonin and norepinephrine), the inflammatory response and environmental factors (infections, metals, magnetic fields, head injury, childhood malnutrition and vitamin deficiencies). Genetic factors have a role in the development of Alzheimer's disease. The major focus of research in late-onset (onset at 65 years or older) Alzheimer's disease has been the gene for apolipoprotein E (ApoE). Other genetic factors for late-onset Alzheimer's disease include mutations in the genes encoding the β-amyloid precursor protein, ubiquitin-B. Research has shown that mutations in the genes presenilin-1 and presenilin-2 account for most cases of early onset (onset at less than 65 years old) Alzheimer's disease.

Most drugs currently used, or under investigation, to treat Alzheimer's disease are aimed at slowing progression; there is no cure. These include cholinergic protective drugs (such as tacrine and donepezil), anti-inflammatories (nonsteroidal anti-inflammatory drugs, corticosteroids, corticotrophin releasing factor, thalidomide and tenidap), oestrogen, antioxidants (such as vitamin E, selegiline and ginkgo biloba), nicotine, propentofylline, hydergine, paclitaxel and CX516.

The present invention seeks to overcome the problem(s) associated with the prior art.

### SUMMARY OF THE INVENTION

The present invention is based on the surprising finding that a deficiency in the retinoid signalling pathway can underlie neurological disorder(s).

According to a first aspect of the present invention there is provided a method for treating a condition in a subject comprising administering an effective amount of an agent to a subject wherein said agent modulates one or more component(s) of the retinoid signalling pathway.

Preferably the condition is a neurological condition such as a motor neurone disease, a cerebral dementing disorder, degenerative movement disorder, or any disorder of the central and/or peripheral nervous system(s).

As used herein, the term motor neurone disease includes amyotrophic lateral sclerosis and other similar disorders. The term cerebral dementing disorder includes Alzheimer's disease and/or frontotemporal dementia and other similar disorders. The term degenerative movement disorder includes Parkinson's disease, Huntington's disease, ataxias and other similar disorders.

The term “subject”, as used herein, relates to an animal. Preferably said animal is a mammal, preferably a human.

The term “agent”, as used herein, may be one or more molecule(s) such as polypeptide(s) or other macromolecule(s). The term agent may also refer to a vector for example, a retroviral vector or a viral vector or similar entities.

The term “modulates”, as used herein, may mean to stimulate, upregulate, downregulate, inhibit, modify, alter or otherwise affect a component of the retinoid signalling pathway.

The term “retinoid signalling pathway”, as used herein, refers to molecules such as signalling molecules or messenger molecules, polypeptides or fragments thereof which are involved in retinoid signal transduction. A component of this pathway is a subset of the entire pathway and may even be a single molecular species. Examples of such components include vitamin A (retinol), provitamin A (β-carotene), one or more enzyme(s) involved in catalysing the metabolism of vitamin A and/or provitamin A or their metabolites (for example, alcohol dehydrogenases, short-chain dehydrogenase/reductases, aldehyde dehydrogenases), metabolites of vitamin A and/or provitamin A (for example, all-trans retinal or 9-cis retinal), cofactors of retinoid dehydrogenases (for example AND or NADPH), retinoid receptor receptors (for example, retinoic acid receptor α or retinoid X receptor α), retinoic acid responsive genes (for example, islet-1, retinoic acid receptor α2, retinoic acid receptor β2 or stromelysin-1) or cofactors of retinoic acid receptors (for example, receptor interacting protein 140 or nuclear receptor co-repressor), or any other entity involved in retinoid signalling.

In one aspect of the invention, the component of the retinoid signalling pathway is an aldehyde dehydrogenase. Preferably the aldehyde dehydrogenase is retinaldehyde dehydrogenase 2 (RALDH-2).

In another aspect of the invention, the component of the retinoid signalling pathway is a retinoid receptor. Preferably the retinoid receptor is retinoic acid receptor α.

Or, in another aspect of the invention, the components of the retinoid pathway are both an aldehyde dehydrogenase and a retinoid receptor.

In another aspect, the present invention provides a method for treating a condition in a subject comprising administering an effective amount of an agent to said subject wherein said agent modulates the expression of one or more component(s) of the retinoid signalling pathway.

Preferably the condition is a neurological condition as mentioned above and discussed in detail below.

Preferably the component of the retinoid signalling pathway is a gene encoding an aldehyde dehydrogenase. Preferably said aldehyde dehydrogenase is a retinaldehyde dehydrogenase 2 (raldh-2).

Preferably the agent comprises raldh-2.

The term “gene”, as used herein, has its natural meaning and may refer to an entire gene, or to a fragment, variant or derivative thereof. The fragment, variant or derivative thereof which may be used in the present invention include the whole ORF or parts of the ORF.

In another aspect the component of the retinoid signalling pathway is a gene encoding a retinoid receptor. Preferably said retinoid receptor gene encodes retinoic acid receptor α. In this aspect, the agent preferably comprises a retinoid receptor gene.

In another aspect the component of the retinoid signalling pathway is a gene encoding a retinoic acid responsive gene. Preferably said retinoic acid responsive gene encodes Islet-1. In this aspect, the agent preferably comprises a retinoic acid responsive gene.

In another aspect, the present invention provides a pharmaceutical composition comprising a RALDH-2 polypeptide or a fragment, variant or derivative thereof, or a polynucleotide encoding the same, and a pharmaceutically acceptable carrier, diluent or excipient therefor.

In another aspect, the present invention provides the use of a RALDH-2 polypeptide or a fragment, variant or derivative thereof, or a polynucleotide encoding the same, in the manufacture of a medicament for the treatment of a neurological condition.

In another aspect, the present invention provides a gene therapy vector comprising a retinoid receptor gene or a fragment, variant or derivative thereof. Preferably the retinoid receptor gene encodes retinoic acid receptor α.

A gene therapy vector may comprise any suitable delivery means such as a retroviral based or viral based particle comprising the gene construct of interest. This is discussed in more detail below.

In another aspect, the present invention provides a gene therapy vector comprising an aldehyde dehydrogenase gene or a fragment, variant or derivative thereof. Preferably the aldehyde dehydrogenase gene encodes raldh-2.

In another aspect, the present invention provides a gene therapy vector comprising a retinoic acid responsive gene or a fragment, variant or derivative thereof. Preferably the retinoic acid responsive gene encodes Islet-1.

In another aspect, the present invention provides a gene therapy vector comprising the mouse or human raldh-2 gene or a fragment, variant or derivative thereof.

### DETAILED DESCRIPTION OF THE INVENTION

In addition to the entire amino acid sequences and nucleotide sequences mentioned herein, the present invention also encompasses the use of one or more fragment(s), variant(s), or derivative(s) of any thereof.

Fragments of a polypeptide or nucleic acid may in theory be almost any size, as long as they retain one characteristic of said parent molecule. Fragments may be truncated forms of the parent molecule, for example they may be truncated at the N-terminus/5′- end, or may be truncated at the C-terminus or 3′-end, or may be truncated from both ends. Fragments may also be produced by shotgun or sonication techniques, which would generally be expected to produce molecules truncated from one or both ends of the parent molecule. Preferably, fragments may be at least 3 amino acids or 9 nucleotides in length.

Derivatives are based on or derived from a reference/parent molecule. A derivative may be a molecule with an internal deletion or truncation with respect to the parent molecule. Derivatives of molecule(s) may also comprise mutants thereof, which may contain amino acid or nucleotide deletions, additions or substitutions, subject to the requirement to maintain at least one feature characteristic of said molecule. This feature could be a functional or structural feature of the parent/reference molecule. A preferred feature retained by a derivative of a parent/reference molecule is homology (ie. sequence identity). Thus, conservative amino acid or nucleotide substitutions may be made substantially without altering the nature of the molecule, as may truncations from the N- or C-terminal ends, or the corresponding 5′- or 3′- ends of a nucleic acid. Deletions or substitutions may moreover be made to the fragments of the molecule(s) comprised by the invention.

Substitution may also be made by unnatural amino acids which include; alpha* and alpha-disubstituted* amino acids, N-alkyl amino acids*, lactic acid*, halide derivatives of natural amino acids such as trifiuorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic acid # and L-Phe (4-benzyl)*. The notation * has been utilised for the purpose of the discussion above (relating to homologous or non-homologous substitution), to indicate the hydrophobic nature of the derivative whereas # has been utilised to indicate the hydrophobic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The term ‘variant’ may also encompass one or more isoform(s), or domain shuffled enzyme(s) or nucleic acids encoding same. With respect to smaller molecules, the term variant will be understood to include analogues, protected or deprotected forms, intermediates and/or salts of the parent /reference molecule.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art (see, for example, “PCR Protocols: A guide to methods and applications”, M. A. Innis, D. H. Gelfand, J. J. Sninsky, T. J. White (eds.). Academic Press, New York, 1990).

The fragments, variants, mutants and other derivatives of the retinoid signalling pathway molecule(s), or nucleic acids encoding them, preferably retain substantial homology with said molecule(s). As used herein, “homology” means that the two entities share sufficient characteristics for the skilled person to determine that they are similar in origin and/or function. Preferably, homology is used to refer to sequence identity. Thus, the derivatives of the molecule preferably retain substantial sequence identity with the sequence of said molecule.

“Substantial homology”, where homology indicates sequence identity, means more than 75% sequence identity and most preferably a sequence identity of 90% or more. Homology comparisons can be conducted by eye, or more usually, with the aid of readily available sequence comparison programs including the BLAST comparison technique which is well known in the art, and is described in Ausubel et al., Short Protocols in Molecular Biology (1999) 4^{th} Ed, John Wiley & Sons, Inc. These commercially available computer programs can calculate % homology between two or more sequences.

Retinoid Signalling Pathway

In accordance with the present invention, a component of the retinoid signalling pathway may be any one or more molecule(s) as discussed below.

There are three major families of enzymes which contribute to the metabolism of the active retinoid forms (Duester, 2000). Alcohol dehydrogenases (ADH) catalyse the reversible oxidation/reduction of retinol and retinal. Known alcohol dehydrogenases, with preferred cofactors shown in brackets, include ADH1 (NAD), ADH2 (NAD), ADH4 (NAD), ADH7 (NAD) and ADH8 (NADPH). Short-chain dehydrogenase/reductases (SDR) catalyse the reversible oxidation/reduction of retinol and retinal. Known short-chain dehydrogenase/reductases with, where applicable, preferred cofactors shown in brackets include RoDH1 (NADP), RoDH2 (NADPH), RoDH3, RoDH4 (NAD), CRAD1 (NAD), CRAD2 (NAD), RDH5 (NAD) and retSDR1 (NADPH). Aldehyde dehydrogenases principally catalyse the oxidation of retinal to retinoic acid. Known aldehyde dehydrogenases, with preferred cofactors shown in brackets, include ALDH1 (NAD), ALDH6 (NAD), RALDH2 (NAD) and ALDH-t (NAD).

RARs and RXRs are ligand-dependent transcription factors that regulate gene expression either by upregulating the expression of genes, by binding to retinoic acid responsive elements present in the promoter, or by downregulating the expression of genes, by antagonising the enhancer action of other transcription factors (Nagpal and Chandraratna, 1998). There are three subtypes of RARs (α, β and γ) which are encoded by separate genes. In addition there are multiple isoforms of each subtype due to alternative splicing and differential promoter use. RARα has two main isoforms (α1 and α2), RARβ has four main isoforms (β1, β2, β3 and β4), RARγ has two main isoforms (γ1 and γ2). There are three subtypes of RXRs (α, β and γ) encoded by separate genes. RXRs are thought to produce various isoforms from a single gene.

RARs and RXRs upregulate gene expression by binding to the promoter regions of retinoid-responsive genes as transcriptionally active RAR-RXR heterodimers Nagpal et al 1992), or as RXR homodimers (Lehmann et al, 1992), or as RXR heterodimers with orphan receptors (Mangelsdorf and Evans, 1995). The retinoic acid-responsive elements (RAREs) of retinoid responsive genes consist of direct repeats of the sequence (consensus) 5′-RGKTCA-3′ (where R is a G or an A and K is a G or a T) separated by two (DR2) or five (DR5) base pair direct repeats (Ross et al, 2000; Kastner et al, 1995; Nagpal and Chandraratna, 1998). The RXR response element is a direct repeat of the same sequence separated by one (DR1) base pair (Kastner et al, 1995; Nagpal and Chandraratna, 1998). Genes containing RAREs include RARβ, RARα, RARγ, CRABPII, CRBPI and CRBPII. Genes containing RXREs include CRABPII, CRBPII, phosphoenolpyruvate carboxykinase, acyl CoA oxidase (ACO), MHC I, Apo A1 and enoyl-CoA hydratase/3-hydroxyacyl-CoA dehydrogenase.

Retinoid induced genes include RAR receptor genes (for example, RARα2, RARβ2 and RARγ2), genes encoding proteins involved in retinoic acid catabolism (for example, cellular retinoic acid binding protein II, cellular retinol binding protein I and cellular retinol binding protein II) and genes involved in retinoic acid synthesis (for example, ADH3). Retinoid repressed genes include genes associated with cell proliferation (for example, Fos, myc, transforming growth factor-β1 and epidermal growth factor receptor), abnormal differentiation (for example, skin-derived anti-leukoproteinase and transglutarninase) and inflammation (for example, stromelysin-1, IL-2 and TNF-α).

Studies have suggested that there are cofactors of retinoic acid receptors which modulate the transcriptional activity of these receptors. These include co-activators, such as receptor interacting protein 140 (RIP140) and thyroid receptor interacting protein 1 (TRIP1), and co-repressors, such as nuclear receptor co-repressor (N-CoR) and silencing mediator for retinoid and thyroid hormone receptors (SMRT) (Nagpal and Chandraratna, 1998). These too may be considered components of the retinoid signalling pathway as defined herein.

Neurological Disorders

The terms neurological condition, neurological disorder and neurological disease are used synonymously or interchangeably herein to refer to a neurological condition such as a motor neurone disease, a cerebral dementing disorder, degenerative movement disorder, or any disorder of the central and/or peripheral nervous system(s). Methods of the present invention may be useful in the treatment of neurological conditions, examples of which are discussed below.

Motor neurone disease such as amyotrophic lateral sclerosis and other similar disorders. Cerebral dementing disorder such as Alzheimer's disease and/or frontotemporal dementia and other similar disorders. Degenerative movement disorders such as Parkinson's disease, Huntington's disease, ataxias and other similar disorders.

Vector Construction

In general, a transgene according to the present invention will comprise an expressed nucleotide sequence, which may be transcribed to RNA and optionally translated to produce a polypeptide, and a vector.

A vector is a tool that allows or facilitates the transfer of an entity from one environment to another. By way of example, some vectors used in recombinant DNA techniques allow entities, such as a segment of DNA (such as a heterologous DNA segment, such as a heterologous cDNA segment), to be transferred into a target cell, Optionally, once within the target cell, the vector may then serve to maintain the heterologous DNA within the cell or may act as a unit of DNA replication. Examples of vectors used in recombinant DNA techniques include plasmids, chromosomes, artificial chromosomes or viruses.

Non-viral delivery systems include but are not limited to DNA transfection methods. Here, transfection includes a process using a non-viral vector to deliver a gene to a target mammalian cell.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotechnology 1996 14; 556), and combinations thereof.

As used herein, “plasmid” refers to discrete elements that are used to introduce heterologous DNA into cells for either expression or replication thereof. Selection and use of such vehicles are well within the skill of the artisan. Many plasmid vectors are available, and selection of appropriate vector will depend on the intended use of the vector, i.e. whether it is to be used for DNA amplification or for DNA expression, the size of the DNA to be inserted into the vector, and the host cell to be transformed with the vector. Each vector contains various components depending on the host cell for which it is compatible. The plasmid vector components generally include, but are not limited to, one or more of the following: an origin of replication, one or more marker genes, an enhancer element, a promoter, a transcription termination sequence, a polyadenylation signal, intronic sequences, a signal sequence and any other sequences necessary to regulate transcription and/or translation.

The term “promoter” is used in the normal sense of the art, e.g. sequences which enable RNA polymerase binding and transcription initiation in the Jacob-Monod theory of gene expression.

The term “enhancer” refers to a DNA sequence which is not necessarily involved directly in transcription initiation, but is capable of enhancing transcription. The positioning of enhancers relative to the promoter is flexible, and enhancers are active in an orientation-independent manner. Enhancers bind to additional components which may interact with the transcription initiation complex and thus upregulate transcription.

Plasmid vectors generally contain nucleic acid sequences that enable the vector to replicate in one or more selected host cells. Typically in cloning vectors, this sequence is one that enables the vector to replicate independently of the host chromosomal DNA, and includes origins of replication or autonomously replicating sequences. Such sequences are well known for a variety of mammalian cells, bacteria, yeast and viruses. The origin of replication from the plasmid pBR322 is suitable for most Gram-negative bacteria, the 2μ plasmid origin is suitable for yeast, and various viral origins (e.g. SV 40, polyoma, adenovirus) are useful for cloning vectors in mammalian cells. Generally, the origin of replication component is not needed for mammalian expression vectors unless these are used in mammalian cells competent for high level DNA replication, such as COS cells.

Most expression vectors are shuttle vectors, i.e. they are capable of replication in at least one class of organisms but can be transfected into another class of organisms for expression. For example, a vector is cloned in E. coli and then the same vector is transfected into cells of the host organism even though it is not capable of replicating independently of the host cell chromosome. DNA may also be replicated by insertion into the host genome. DNA can be amplified by PCR and be directly transfected into the host cells without any replication component.

Advantageously, an expression (and cloning) vector may contain a selection gene also referred to as selectable marker. This gene encodes a protein necessary for the survival or growth of transformed host cells grown in a selective culture medium. Host cells not transformed with the vector containing the selection gene will not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available from complex media.

The following markers, for example, have been used successfully in, inter alia, retroviral vectors. The bacterial neomycin and hygromycin phosphotransferase genes which confer resistance to G418 and hygromycin respectively (Palmer et al 1987 Proc Natl Acad Sci 84: 1055-1059; Yang et al 1987 Mol Cell Biol 7: 3923-3928); a mutant mouse dihydrofolate reductase gene (dhfr) which confers resistance to methotrexate (Miller et al 1985 Mol Cell Biol 5: 431-437); the bacterial gpt gene which allows cells to grow in medium containing mycophenolic acid, xanthine and aminopterin (Mann et al 1983 Cell 33: 153-159); the bacterial hisD gene which allows cells to grow in medium without histidine but containing histidinol (Danos and Mulligan 1988 Proc Natl Acad Sci 85: 6460-6464); the multidrug resistance gene (mdr) which confers resistance to a variety of drugs (Guild et al 1988 Proc Natl Acad Sci 85: 1595-1599; Pastan et al 1988 Proc Natl Acad Sci 85: 4486-4490) and the bacterial genes which confer resistance to puromycin or phleomycin (Morgenstern and Land 1990 Nucleic Acid Res 18: 3587-3596).

All of these markers are dominant selectable markers and allow chemical selection of most cells expressing these genes. β-galactosidase can also be considered a dominant marker; cells expressing β-galactosidase can be selected by using fluorescence-activated cell sorting (FACS). In fact, any cell surface protein can provide a selectable marker for cells not already making the protein. Cells expressing the protein can be selected by using the fluorescent antibody to the protein and a cell sorter. Other selectable markers that have been included in vectors include the hprt and HSV thymidine kinase which allows cells to grow in medium containing hypoxanthine, amethopterin and thymidine.

Since the replication of vectors is conveniently done in E. coli, an E. coli genetic marker and an E. coli origin of replication are advantageously included. These can be obtained from E. coli plasmids, such as pBR322, Bluescript© vector or a pUC plasmid, e.g. pUC18 or pUC19, which contain both E. coli replication origin and E. coli genetic marker conferring resistance to antibiotics, such as ampicillin.

Suitable selectable markers for mammalian cells are those that enable the identification of cells competent to take up a vector containing the transgene, such as dihydrofolate reductase (DHFR, methotrexate resistance), thymidine kinase, or genes conferring resistance to G418 or hygromycin. The mammalian cell transformants are placed under selection pressure which only those transformants which have taken up and are expressing the marker are uniquely adapted to survive. In the case of a DHFR or glutamine synthase (GS) marker, selection pressure can be imposed by culturing the transformants under conditions in which the pressure is progressively increased, thereby leading to amplification (at its chromosomal integration site) of both the selection gene and the linked transgene DNA. Amplification is the process by which genes in greater demand for the production of a protein critical for growth, together with closely associated genes which may encode a desired protein, are reiterated in tandem within the chromosomes of recombinant cells. Increased quantities of desired protein are usually synthesised from thus amplified DNA.

Expression and cloning vectors usually contain a promoter that is recognised by the host organism and is operably linked to the transgene. Such a promoter may be inducible or constitutive. The promoters are operably linked to the transgene by removing the promoter from the source DNA and inserting the isolated promoter sequence into the vector. Both the native promoter sequence usually associated with the transgene in nature, if applicable, and many heterologous promoters may be used to direct amplification and/or expression of the transgene. The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences.

Transgene transcription from vectors in mammalian hosts may be controlled by promoters derived from the genomes of viruses such as polyoma virus, adenovirus, fowlpox virus, bovine papilloma virus, avian sarcoma virus, cytomegalovirus (CMV), a retrovirus and Simian Virus 40 (SV40), from heterologous mammalian promoters such as the actin promoter or a very strong promoter, e.g. a ribosomal protein promoter, and from the promoter normally associated with the coding sequence of the transgene, provided such promoters are compatible with the host cell systems.

Transcription of the transgene by higher eukaryotes may be increased by inserting an enhancer sequence into the vector. Enhancers are relatively orientation and position independent. Many enhancer sequences are known from mammalian genes (e.g. elastase and globin). However, typically one will employ an enhancer from a eukaryotic cell virus. Examples include the SV40 enhancer on the late side of the replication origin (bp 100-270) and the CMV early promoter enhancer. The enhancer may be spliced into the, vector at a position 5′ or 3′ to the transgene, but is preferably located at a site 5′ from the promoter.

Advantageously, a eukaryotic expression vector encoding the transgene may comprise a locus control region (LCR). LCRs are capable of directing high-level integration site independent expression of transgenes integrated into host cell chromatin, which is of importance especially where the transgene is to be expressed in the context of a permanently-transfected eukaryotic cell in which chromosomal integration of the vector has occurred, in vectors designed for gene therapy applications or in transgenic animals.

Vectors may be designed for precise integration into defined loci of the host genome, thus avoiding the disadvantages of random integration. Alternatively, artificial mammalian chromosomes may be used to deliver the genes of interest, thus avoiding any integration-related issues.

Eukaryotic expression vectors will also contain sequences necessary for the termination of transcription and for stabilising the mRNA. Such sequences are commonly available from the 5′ and 3′ untranslated regions of eukaryotic or viral DNAs or cDNAs. These regions may contain nucleotide segments which direct polyadenylation of the messenger RNA during post-transcriptional processing thereof.

An expression vector includes any vector capable of expressing nucleic acids that are operatively linked with regulatory sequences, such as promoter regions, that are capable of expression of such DNAs. Thus, an expression vector refers to a recombinant DNA or RNA construct that, upon introduction into an appropriate host cell, results in expression of the cloned DNA. Appropriate expression vectors are well known to those with ordinary skill in the art and include those that are replicable in eukaryotic and/or prokaryotic cells and those that remain episomal or those which integrate into the host cell genome. For example, nucleic acids encoding a transgene may be inserted into a vector suitable for expression of cDNAs in mammalian cells, e.g. a CMV enhancer-based vector such as pEVRF (Matthias, et al., (1989) NAR 17, 6418).

The promoter and enhancer of the transgene are preferably strongly active, or capable of being strongly induced, in the primary target cells under conditions for production of the gene product of interest. The promoter and/or enhancer may be constitutively efficient, or may be tissue or temporally restricted in their activity.

Other preferred additional components include entities enabling efficient expression of a transgene or a plurality of transgenes.

One method of regulating the expression of such components is by using the tetracycline on/off system described by Gossen and Bujard (1992 Proc Natl Acad Sci 89: 5547) as described for the production of retroviral gal, pol and VSV-G proteins by Yoshida et-al (1997 Biochem Biophys Res Comm 230: 426).

Construction of vectors according to the invention employs conventional ligation techniques. Isolated plasmids or DNA fragments are cleaved, tailored, and religated in the form desired to generate the plasmids required. If desired, analysis to confirm correct sequences in the constructed plasmids is performed in a known fashion. Suitable methods for constructing expression vectors, preparing in vitro transcripts, introducing DNA into host cells, and performing analyses for assessing expression and function are known to those skilled in the art. Gene presence, amplification and/or expression may be measured in a sample directly, for example, by conventional Southern blotting, Northern blotting to quantitate the transcription of mRNA, dot blotting (DNA or RNA analysis), or in situ hybridisation, using an appropriately labelled probe.

Suitable techniques are fully described in the literature. See for example; Sambrook et al (1989) Molecular Cloning; a laboratory manual; Hames and Glover (1985-1997) DNA Cloning: a practical approach, Volumes I-IV (second edition); Methods for the engineering of immunoglobulin genes are given in McCafferty et al (1996) “Antibody Engineering: A Practical Approach”.

Those skilled in the art will readily envisage how these methods may be modified, if desired.

Viral vector systems include but are not limited to adenovirus vectors, adeno-associated viral (AAV) vectors, herpes viral vectors, retroviral vectors, lentiviral vectors and baculoviral vectors.

Viral vectors according to the present invention are preferably retroviral vectors. The term “retroviral vector” typically includes a retroviral nucleic acid which is capable of infection, but which is not capable, by itself, of replication. Thus it is replication defective. A retroviral vector typically comprises one or more transgene(s), preferably of non-retroviral origin, for delivery to target cells. A retroviral vector may also comprise a functional splice donor site (FSDS) and a functional splice acceptor site (FSAS) so that when the FSDS is upstream of the FSAS, any intervening sequence(s) are capable of being spliced. A retroviral vector may comprise further non-retroviral sequences, such as non-retroviral control sequences in the U3 region which may influence expression of a transgene(s) once the retroviral vector is integrated as a provirus into a target cell. The retroviral vector need not contain elements from only a single retrovirus. Thus, in accordance with the present invention, it is possible to have elements derivable from two of more different retroviruses or other sources

The term “retroviral pro-vector” typically includes a retroviral vector genome as described above but which comprises a first nucleotide sequence (NS) capable of yielding a functional splice donor site (FSDs) and a second NS capable of yielding a functional splice acceptor site (FSAS) such that the first NS is downstream of the second NS so that splicing associated with the first NS and the second NS cannot occur. Upon reverse transcription of the retroviral pro-vector, a retroviral vector is formed.

The term “retroviral vector particle” refers to the packaged retroviral vector, that is preferably capable of binding to and entering target cells. The components of the particle, as already discussed for the vector, may be modified with respect to the wild type retrovirus. For example, the Env proteins in the proteinaceous coat of the particle may be genetically modified in order to alter their targeting specificity or achieve some other desired function.

The retroviral vector of this aspect of the invention may be derivable from a murine oncoretrovirus such as MMLV, MSV or MMTV; or may be derivable from a lentivirus such as HIV-1 or EIAV; or may be derivable from another retrovirus.

The retroviral vector of the invention can be modified to render the natural splice donor site of the retrovirus non-functional.

The term “modification” includes the silencing or removal of the natural splice donor. Vectors, such as MLV based vectors, which have the splice donor site removed are known in the art. An example of such a vector is pBABE (Morgenstern et al 1990 ibid).

Transgene Construction

In accordance with the present invention, the transgene can be any suitable nucleotide sequence. For example, the sequence may be DNA or RNA—which may be synthetically prepared or may be prepared by use of recombinant DNA techniques or may be isolated from natural sources or may be combinations thereof The sequence may be a sense sequence or an antisense sequence. There may be a plurality of sequences, which may be directly or indirectly joined to each other, or combinations thereof.

Suitable transgene coding sequences include those that are of therapeutic and/or diagnostic application such as, but are not limited to: retinoid acid receptors, alcohol dehydrogenases, short-chain dehydrogenase/reductase, aldehyde dehydrogenases, retinoid responsive genes and derivatives thereof (such as with an associated reporter group). When included, such coding sequences may be typically operatively linked to a suitable promoter, which may be a promoter driving expression of a ribozyme(s), or a different promoter or promoters.

The transgene may encode a fusion protein or a segment of a coding sequence.

The delivery of one or more therapeutic genes according to the present invention may be used alone or in combination with other treatments or components of the treatment.

For example, the methods of the present invention may be used to deliver one or more transgene(s) useful in the treatment neurological disorders such as motor neurone diseases such as amyotrophic lateral sclerosis, cerebral dementing disorders such as Alzheimer's disease and/or frontotemporal dementia, degenerative movement disorders such as Parkinson's disease, Huntington's disease and atdxias, and multiple sclerosis, or other neurological condition.

Transformation of Cells

Cell populations for use according to the invention may be transformed by any appropriate technique suitable for introduction of nucleic acids into cells, for example as set forth in the general literature referred to above.

Cell populations according to the present invention preferably comprise neuronal cells.

A vector comprising a transgene according to the invention may be introduced into the cell population by any suitable means.

Pharmaceutical Compositions

The present invention also provides a pharmaceutical composition comprising a therapeutically effective amount of the agent(s) of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of the present invention may be administered alone but will generally be administered as a pharmaceutical composition for example, when the component(s) is/are in admixture with a suitable pharmaceutical excipient, diluent or carrier selected with regard to the intended route of administration and standard pharmaceutical practice.

For example, the components can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

It is to be understood that not all of the components of the pharmaceutical need be administered by the same route. Likewise, if the composition comprises more than one active component, then those components may be administered by different routes.

If an agent of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art

The component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

Depending upon the need, the agent may be administered at a dose of from 0.01 to 30 mg/kg body weight, such as from 0.1 to 10 mg/kg, more preferably from 0.1 to 1 mg/kg body weight.

If the composition is applied topically, then typical doses may be in the order of about 1 to 50 mg/cm² of tissue.

Formulation

The component(s) of the present invention may be formulated into a pharmaceutical composition, such as by mixing with one or more of a suitable carrier, diluent or excipient, by using techniques that are known in the art

Pharmaceutically Active Salt

The agent of the present invention may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Animal Test Models

In vivo models may be used to investigate and/or design therapies or therapeutic agents to treat neurological disorders. The models could be used to investigate the effect of various tools/lead compounds on a variety of parameters which are implicated in the development of or treatment of a neurological disorder. The animal test model will be a non-human animal test model.

In another embodiment, the invention relates to the use of RAR alpha agonist to increase the production of CHAT, thereby increasing acetylcholine production, is disclosed. Acetylcholine is the neurotransmitter lost in alzheimers disease.

In another embodiment, the invention relates to the repair of cholinergic neuron(s) via gene therapy with RAR alpha is disclosed.

In another embodiment, the invention relates to the use of stem cells transfected with RAR alpha for transplant into the adult brain.

It is known that strokes can be associated with beta amyloid deposition (eg. Vidal et al, Acta Neuropathologica volume 100 issue 2000 pp 1-12: “Senile dementia associated with amyloid beta protein angiopathy and tau perivascular pathology but not neuritic plaques in patients homozygous for the APOE epsilon 4 allele.”) We disclose herein method(s) for addressing the retinoid signalling involved in such deposition. Thus, in another embodiment, the invention relates to use of RAR alpha agonists in treatment and/or prevention of strokes associated with beta amyloid deposition.

### EXAMPLES

The invention will now be described by way of example with reference to the figures described below. The following examples are offered by way of illustration and are not intended in any way to limit the scope of the invention.

### BRIEF DESCRIPTION OF FIGURES

FIG. 1. Effect of a retinoid deficient diet on adult rats. A. 6 month old normal fed rat, B. 6 month old retinoid deficient rat, C. 1 year old retinoid deficient rat. Normal rats (A) extend their hindlimbs when held by the tail, whereas retinoid deficient rats retract their hindlimbs (B and C).

FIG. 2. Expression of NF200 in cervical (A and C) and lumbar cord (B and D). A. Cervical cord of 6 month old normal fed rat, B. lumbar cord of 6 month old normal fed rat, C. cervical cord of 6 month old retinoid deficient rat, D. lumbar cord of 6 month old retinoid deficient rat.

FIG. 3. Reactive astrocytosis in the lumbar cord. Expression of GFAP in astrocytes. A. normal lumbar cord, B. retinoid deficient lumbar cord.

FIG. 4. Expression of RARα in motor neurones of the adult rat. A. lumbar cord of 6 month old retinoid deficient rat. B. lumbar cord of 6 month old normal fed rat

FIG. 5. Graph of percentage of motor neurones in the lumbar cord expressing islet-1 and components of the retinoid signalling pathway in age matched normal and motor neurone diseased patients. Columns: 1. islet-1 positive motor neurones in normal cord, 2. islet-1 positive motor neurones in diseased cord 3. RARα positive motor neurones in normal cord, 4. RARα positive motor neurones in diseased cord. 5. raldh-2 positive motor neurones in normal cord, 6. Raldh-2 positive motor neurones in diseased cord.

FIG. 6. Expression of islet-1 and components of the retinoid signalling pathway by in situ hybridisation in the lumbar cord of normal (A, C and E) and an age matched patient suffering from spontaneous motor neurone disease (B, D and F). A, B. islet-1 expression, C, D. RARα expression, E, F. raldh-2 expression. There is a down regulation of each of the three transcripts in the diseased patient compared to the age matched non-diseased patient.

FIG. 7. β-galatosidase activity in the adult brain of a RARElacZ reporter mouse. A. Low power view of a sagittally sectioned brain showing three regions of strong reporter activity. These three regions are: h=hippocampus; c=choroid plexus; p=Purkinje cells of the cerebellum. Each of these is shown at higher power in B-D. B. Reporter activity in the hippocampus. C. Reporter activity in the choroid plexus of the lateral ventricle. D. Reporter activity on the cerebellum showing a blue line which is the Purkinje cells.

FIG. 8. HPLC chromatograms of the retinoids present in 3 parts of the brain.

FIG. 9. Expression of enzymes, binding proteins and receptors in the adult mouse cerebellum. A. RALDH2 immunoreactivity in the meninges (arrowhead) and capillary linings (arrow). B. RALDH2 immunoreactivity in the choroid plexus of the fourth ventricle. C. RALDH3 in situ hybridisation showing expression only in the Purkinje cells (arrowheads). D. CRBP I immunoreactivity in the meninges (arrowhead). E. CRBP I immunoreactivity in the choroid plexus of the fourth ventricle. F. RARα in situ hybridisation showing strong expression in the Purkinje cells (arrowheads) and weak expression in the granule cell layer below the Purkinje cells. G. RARβ in situ hybridisation showing absence of expression in the cerebellum (arrowheads=Purkinje cells). H. RXRα in situ hybridisation showing strong expression in the Purkinje cells (arrowheads) and weak expression in the granule cell layer below the Purkinje cells. I. RXRγ in situ hybridisation showing strong expression in the Purkinje cells (arrowheads) and weak expression in the molecular layer above the Purkinje cells. ml=molecular layer; gl=granule cell layer.

FIG. 10. Changes in the expression patterns of receptors and enzymes and Purkinje cells in normal and vitamin A deficient rat cerebellum. A. Normal expression of RARα in the cerebellum of a one year old rat showing expression in the Purkinje cells (arrowhead) and the granule cell layer below. B. Expression of RARα in the cerebellum of a vitamin A-deficient one year old rat showing complete down regulation of this gene. C. Normal expression of CYP26 in the cerebellum of a control one year old rat showing weak expression in the Purkinje cells (arrowhead) and strong expression in the granule cell layer below. D. Expression of CYP26 in the cerebellum of a vitamin A-deficient one year old rat showing complete down regulation of this gene. E & F. Calbindin staining of a normal one year old rat cerebellum showing Purkinje cells and their dendrites. G & H. Calbindin staining of a vitamin A-deficient one year old rat cerebellum showing a massive reduction in Purkinje cell numbers and the absence of dendrites in those that are remaining.

FIG. 11. Purkinje cell counts in 6 month old normal and vitamin A-deficient and one year old normal and vitamin A-deficient cerebella. Column 1=normal 6 month old rats (n=4); column 2=normal 1 year old rats (n=2); column 3=6 month old vitamin A-deficient rats (n=2); column 4=1 year old vitamin A-deficient rat. The 6 month old vitamin A deficient counts are significantly different from both 6 month old and 1 year old control counts (p>0.0001). The cell counts on each brain were repeated 12 times.

FIG. 12. Summary diagram of the expression patterns in a representation of the cerebellum. Red=RA. Arrows represent a potential supply of RA from the meninges and capillaries and the red Purkinje cells are depicted as containing RA because of the presence of RALDH3 and the RARElacZ reporter result (FIG. 7D).

FIG. 13—Expression of B amyloid in the brain of 1 year adult rats
A. control rat-normal rat brain. One year old. Amyloid staining brown
B. retinoid deficient rat—RA deficient rat brain. One year old. Amyloid staining brown
   retinoid deficient rat—RA deficient rat brain. One year old. Amyloid staining brown.

FIG. 14—Expression of RARα in cerebral cortex.

FIG. 15—Expression of CHAT.

FIG. 16—Expression of RALDH-2 in cholinergic neurons.

FIG. 17. HPLC analysis of retinoids in normal human lumbar spinal cord (A and B) and human lumbar motoneuron diseased samples (C and D). Arrow heads denote retinoic acids present in the normal human lumbar cord which are absent in the diseased samples.

FIG. 18. HPLC analysis of retinoids in human Alzheimer's diseased (A) and non diseased cerebral cortex (B). Note that there are a number of novel retinoids in both samples, and that in the diseased samples there is a loss of retinoic acid compared to the samples from normal brains.

### EXAMPLE 1

### Vitamin A Depletion Induces Motor Neurone Degeneration in Adult Rats

This example shows that a dietary retinoid defect gives rise to a downregulation of retinoic acid receptor α expression and there is motor neurone degeneration.

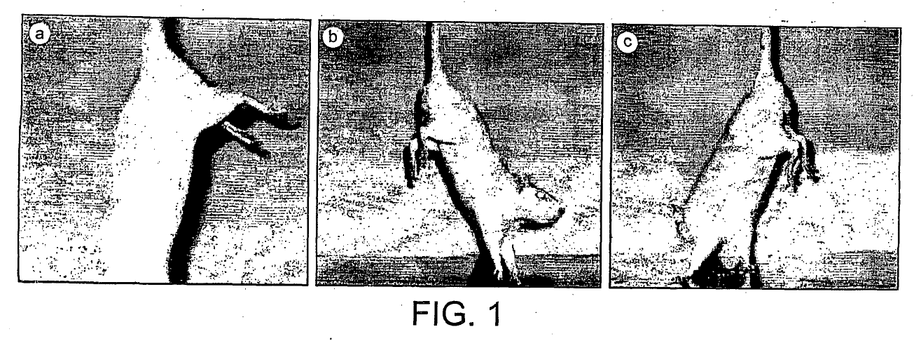

Weaned rats (Wistar) are fed on a normal diet (controls) or a commercially available vitamin A-free diet (Special Diet Services) ad libidum. After 6 months of a retinoid deficient diet the rats are distinguished from normal fed rats by muscle atrophy and hindlimb retraction when held by the tail (FIGS. 1a & b). These phenotypes are more severe after 1 year of a retinoid deficient diet (FIG. 1c).

Rats are killed by perfusion with 4% paraformaldehyde/0.5% glutaraldehyde and the tissues prepared for in situ hybridisation and immunohistochemistry. HPLC measurements of liver tissue show that rats on a vitamin A-free diet are vitamin A-depleted after 6 months and virtually vitamin A-deficient after 1 year.

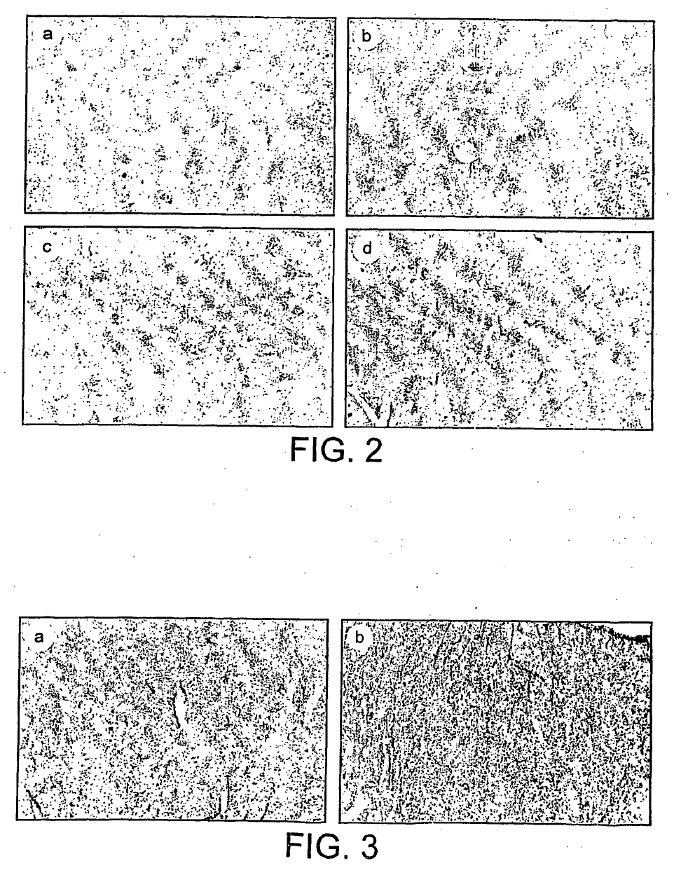

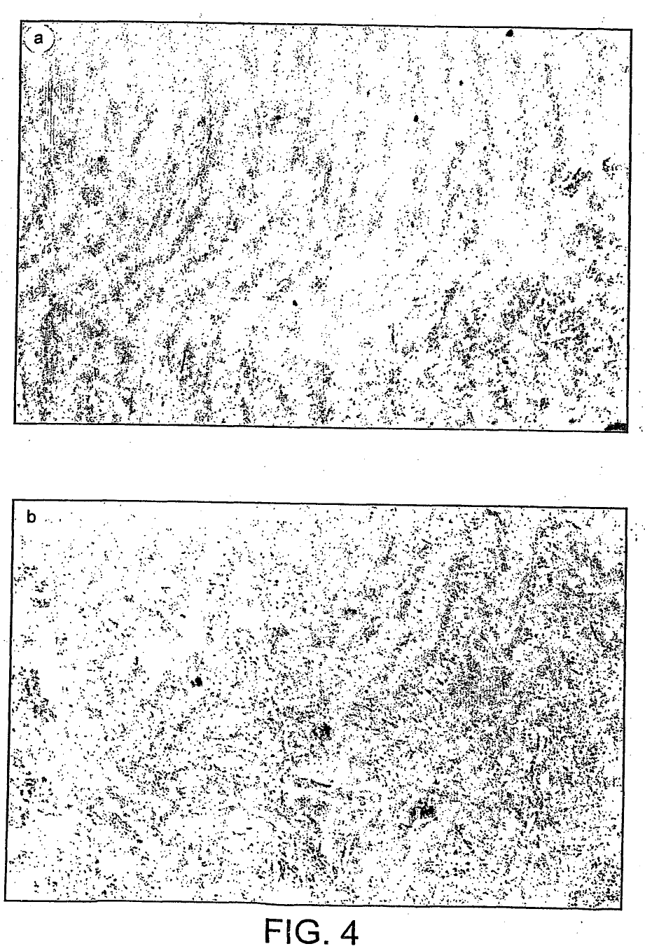

In situ hybridisation and immunohistochemistry is carried out as described by Corcoran et al (2000) using the mouse RARα probe and the NF200 antibody (obtained from sigma). The number of positive motor neurones is counted on whole chord sections. In the retinoid deficient rat the motor neurones of the lumbar cord have more vacuolar lesions (FIG. 2d) than the motor neurones located in the cervical cord (FIG. 2c). In the normal rat no vacuolar lesions are seen in the motor neurones at either level of the spinal cord examined (FIGS. 2a & b). Sections stained for NF200 show that there is an accumulation of this neurofilament in the cell body of the motor neurones of the lumbar and cervical retinoid deficient cords (FIGS. 2c & d) compared to the same regions of the cord of the normal rat (FIGS. 2a & b). There is also in the retinoid deficient lumbar cord an accumulation of the neurofilament in the axons, some axonal swelling and vacuolation of the axons (FIG. 2d). There is also an increase in reactive astrocytosis in the lumbar cord of the retinoid deficient rats (FIG. 3b) compared with the lumbar cord of the normal rat (FIG. 3a). In situ hybridisation shows that there is a loss of RARα in the motor neurones of the lumbar cord of the retinoid deficient rats compared to the cervical cord (FIGS. 4a & b).

### EXAMPLE 2

### Components of the retinoid signalling pathway are perturbed in the neurones of motor neurone disease patients

This example shows the defects in the retinoid signalling pathway in the motor neurones of patients suffering motor neurone disease.

Post-mortem lumbar spinal cord tissue is obtained cases of spontaneous motor neurone disease and age matched controls. The tissue is fixed in 4% PFA, wax embedded and 10 μM sections cut. The amount of motor neurone loss is assessed by counting the total number of motor neurones. In diseased patients there are fewer neurones compared to the non diseased age matched controls.

In situ hybridisation is carried out as described by Corcoran et al (2000) using the rat islet-1, mouse RARα and mouse raldh-2 probes. The number of positive motor neurones is counted on each whole chord section.

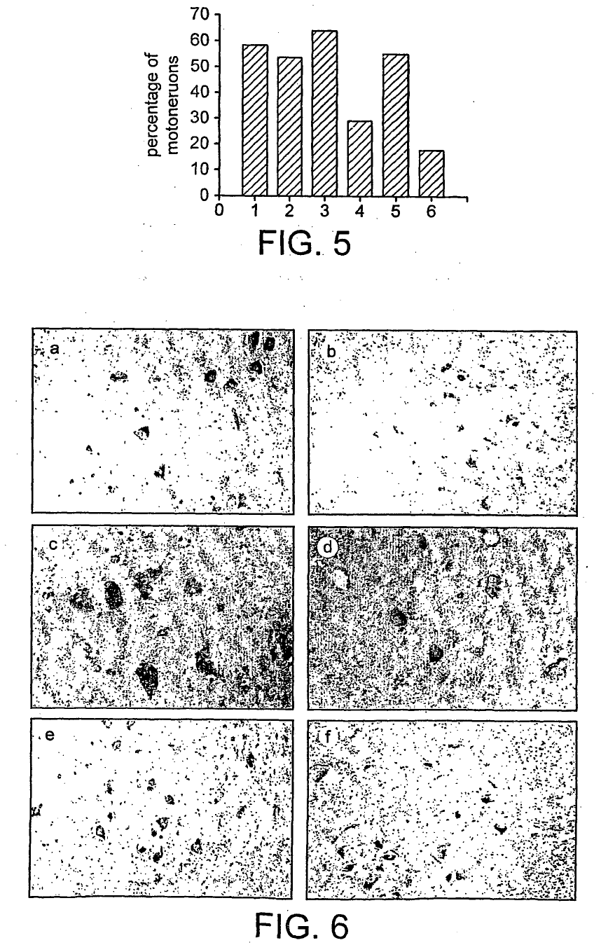

In situ hybridisation shows that there is a decrease in the number of islet-1 motor neurones in the diseased compared to the non-diseased patients but the percentage of motor neurone expressing islet-1 is approximately the same (FIG. 5, columns 1 & 2). However, the level of islet-1 expression in individual motor neurones is lower in the motor neurone diseased patients compared to the normal patients (FIGS. 6a & b). Expression of the RARα receptor is downregulated in the motor neurone diseased patients compared to age matched controls (FIGS. 6c & d). Furthermore the absence of expression of the RARα receptor occurs in more of the motor neurones in motor neurone disease patients than compared to normal samples (FIG. 5, columns 3 & 4). In non-diseased patients more motor neurones express the raldh-2 enzyme (FIG. 5, column 5) than compared to motor neurone diseased patients (FIG. 5, column 6). In the surviving motor neurones of diseased patients expression of raldh-2 is also downregulated compared to non-diseased patients (FIGS. 6e & f).

### EXAMPLE 3

### Modulation of Retinoid Signalling in Adult Nervous System

Summary

The distribution of the signalling molecule retinoic acid (RA) and its molecular transducers (synthetic enzymes, cytoplasmic binding proteins, nuclear receptors) in the adult mouse and rat brain are demonstrated. Using a RARElacZ transgenic reporter mouse we find that the hippocampus, choroid plexus and Purkinje cells of the cerebellum are sites of active RA signalling. By HPLC we find that the cerebellum, but not the rest of the brain, contains high levels of all-trans-RA. The meninges surrounding the cerebellum and the choroid plexus express the enzyme RALDH2 and the binding protein CRBP I whereas the Purkinje cells express the enzyme RALDH3. The Purkinje cells also express the nuclear receptors RARα, RXRα, RXRβ and RXRγ, but not RARβ or RARγ. In order to demonstrate that the Purkinje cells require a continual supply of RA for their functioning and survival we deprive rats of vitamin A in their diet for up to 1 year. After 6 months there is a decline in the expression of the nuclear receptors and the Purkinje cell number. After 1 year there is a complete loss of receptor expression and at least one of the experimental animals shows symptoms of ataxia with a staggering gait and has lost 80% of its Purkinje cells. These data illustrate the requirement for RA signalling in the maintenance of the Purkinje cells as disclosed herein. Thus, it is demonstrated that modulation of this signalling pathway provides therapeutic approaches to ataxia in the human population.

Background

Retinoic acid (RA), the biologically active metabolite of vitamin A, is known to be an important signalling molecule in the developing embryo. RA functions as such because it is rapidly diffusable and can spread across a field of embryonic cells and then acts at the level of the nucleus to switch on or off key developmental genes by binding to ligand activated transcription factors known as retinoic acid receptors (RARs) and retinoid X receptors (RXRs) (1, 2). There are three RARs α, β and γ and three RXRs α, β and γ which form heterodimers and can bind to retinoic acid response elements (RAREs) in the enhancer sequences of retinoic acid responsive genes.

RA itself may be generated from vitamin A (retinol) by the action of two classes of enzymes. Firstly, the retinol/alcohol dehydrogenases which oxidise retinol to retinaldehyde and secondly, the retinaldehyde dehydrogenases which oxidise retinaldehyde to all-trans-RA and 9-cis-RA (3). All-trans-RA is further metabolised by the action of a cytochrome P450 enzyme, CYP26, to products such as 4-oxo-RA, 4-OH-RA and 18-OH-RA (4, 5).

The role of RA in the embryo has been investigated by a variety of means including determination of the distribution of RA itself, examining the expression domains of these enzymes and receptors, increasing or decreasing the supply of RA and overexpressing or knocking out the enzymes and receptors. Such studies have identified key roles for RA in the developing CNS (6), lung (7), limb (8, 9) and kidney (10). In the developing CNS for example, RA is crucial for the development of the hindbrain, the survival of the neural crest and is required for neurite outgrowth (11, 12).

In adult systems, although the skin was identified as a site of drastic alteration in the absence of retinoids in the 1920s (13), little is known about RA signalling in other tissues or organs, or even whether such signalling occurs. In addition to the skin, it is known that vision, haematopoeisis and the immune system and spermatogenesis are deleteriously affected by a lack of vitamin A in the diet. Virtually nothing is known about the retinoid requirement of the adult CNS for its functioning and maintenance, despite its crucial role in CNS development.

Disclosed herein are sites of retinold synthesis and activity in the adult rodent brain, and consequences of the absence of RA. In one approach to the identification of sites of retinoid activity, we disclose the use of a transgenic mouse strain which expresses a RARElacZ transgene. At least three such sites in the adult brain are identified, the hippocampus, the choroid plexus and the Purkinje cells of the cerebellum.

It is then determined whether retinoids can be detected by HPLC in brains separated into three parts. High levels of all-trans-RA are detected only in the cerebellum. In order to reveal which cells in the cerebellun synthesise and use this all-trans-RA we examine the distribution of the enzymes, binding proteins and receptors, which gives us important information about the paracrine/autocrine functioning of RA.

We then disclose the consequences for these active sites in the absence of RA. This is investigated by depriving rats of vitamin A in the diet. The surprising result of this experiment is that the Purkinje cells of the cerebellum disappear resulting in, inter alia, locomotor difficulties. It is thus demonstrated that a lack of vitamin A in the diet, or a malfunctioning retonoid signalling protein or synthetic enzyme, could be responsible for the development of certain types of ataxia which are known to be caused by a loss, failure or inhibition of Purkinje cell functioning. Aspects of the present invention are based on this surprising finding.

### GENERAL METHODS

The generation and use of the RARElacZ transgenic strain has been described previously for use in embryological studies (20). Here the adult brain is fixed in 4% paraformaldehyde overnight, washed in PBS and then stained for β-galactosidase as a wholemount or as slices.

For HPLC studies, retinoids are extracted from the tissue according to the method of Thaller & Eichele (21) by collecting 200-500 mg of lungs and homogenising in 1 ml of stabilising solution (5 mg/ml ascorbic acid, Na₃EDTA in PBS, pH 7.3). The homogenate is extracted twice with 2 volumes of 1:8 methyl acetate/ethyl acetate, with butylated hydroxytoluene as an anti-oxidant, and then dried down under nitrogen. The extract is resuspended in 100 μl methanol, centrifuged at high speed to remove any particulate matter and placed into an autosampler vial for analysis.

Reverse phase HPLC is performed using a Beckman System Gold Hardware with a photodiode array detector and a 5μ C₁₈ LiChrocart column (Merck) with an equivalent precolumn. The mobile phases used are those of Achkar et al. (22) which allow a good separation of the retinoic acids and the retinols. The flow rate is 1.5 ml/min using a gradient of acetonitrile/ammonium acetate (15 mM, pH 6.5) from 40% to 67% acetonitrile in 35 min followed by 100% acetonitrile for an additional 25 min. Individual retinoids are identified according to their uv absorption spectra.

Immunocytochemistry is performed on wax embedded sections fixed in 4% paraformaldehyde, 2% trichloroacetic acid, 20% isopropyl alcohol using a CRBP I antibody (23), a CRABP I antibody (24), a RALDH2 antibody (25) and a calbindin antibody (Sigma). Immunoreactivity is visualised with the avidin-biotinylated peroxidase technique with a kit from Vector Laboratories.

In situ hybridisation is performed on wax embedded sections according to a previously described protocol (26) using RAR and RXR probes synthesised from the appropriate cDNAs (27, 28). The CYP26 probe is from the chicken (29).

For the vitamin A deficiency studies, weaned Wistar female rats are divided into two groups. One group is placed on a normal diet and the other group is fed a commercially available vitamin A-free diet (Special Diet Services). Animals are taken from control and deficient groups at 6 months and 12 months after the initiation of the experiment and perfused with 4% paraformaldehyde, 0.5% glutaraldehyde. Wax sections of the brain are prepared and used for immunocytochemistry and in situ hybridisation as described above.

Retinoid Activity in the Adult Brain

Sites of retinoid activity in the adult brain are disclosed. The surprising identification of such sites permits the design and development of therapeutic approaches to disease states and/or conditions which are implicated in these areas of adult brain. In particular, this example relates to the targetting of cerebral ataxia

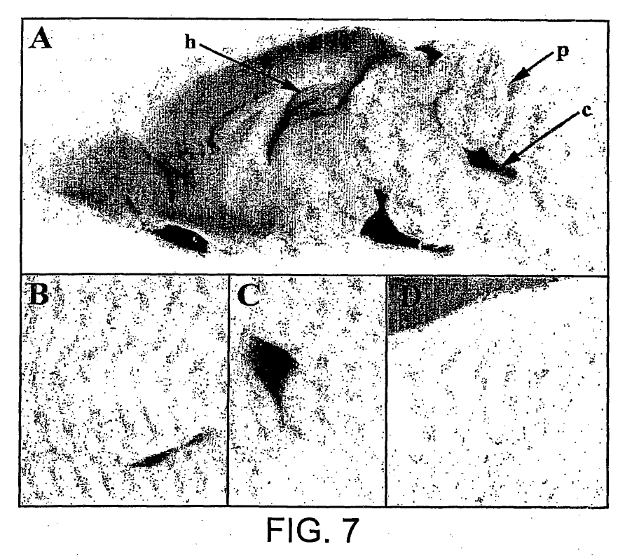

The RARElacZ transgenic mouse strain (20) is used to determine whether there are any active sites of RA activity in the adult brain. Due to the lacZ gene, if the RARE is activated then tissues stain blue. In sagittally sectioned adult mouse brains three areas of RA activity are present (FIG. 7A). One is in the hippocampus (h in FIG. 7A and at high power in FIG. 7B). The second is the choroid plexus (c in FIG. 7A) which is shown in the fourth ventricle in FIG. 7A and at high power in the lateral ventricle in FIG. 7C. The third is in the cerebellum (p in FIG. 7A) which at high power is revealed to be in the Purkinje cells (FIG. 7D).

Endgenous Retinoids in the Adult Mouse Brain

There have been very few attempts to measure retinoids in the adult brain, and no measurements of retinoids in individual parts of the brain have been made in the prior art. Disclosed herein are the identities and locations of numerous retinoid species in different areas of the adult brain. Based on these discoveries, the invention provides candidate effectors and/or targets for modulation of retinoid signalling in the adult brain.

In order to better reveal how the RARElacZ reporter results above relate to endogenous bioactive retinoids, mouse brains are divided into 3 parts and the retinoid content of each is examined. The three parts are i) cerebrum ii) brain stem iii) cerebellum. The retinoids are extracted from 300-500 mg of tissue and separated by reverse phase chromatography. Each experiment is repeated 6-10 times.

As a whole, the retinoid content of these brain sample is highly consistent and shows at least two unique features compared to other adult tissue we have analysed such as lung, kidney, liver etc.

Firstly, there are extremely low levels of all-trans-retinol: cerebrum=9.2±2.3 pg/mg tissue; brainstem=13.5±4.1 pg/mg; cerebellum=38.9±25.5 pg/mg. This is in comparison to lungs which contains 5156 pg/mg, about 100-500× more (30).

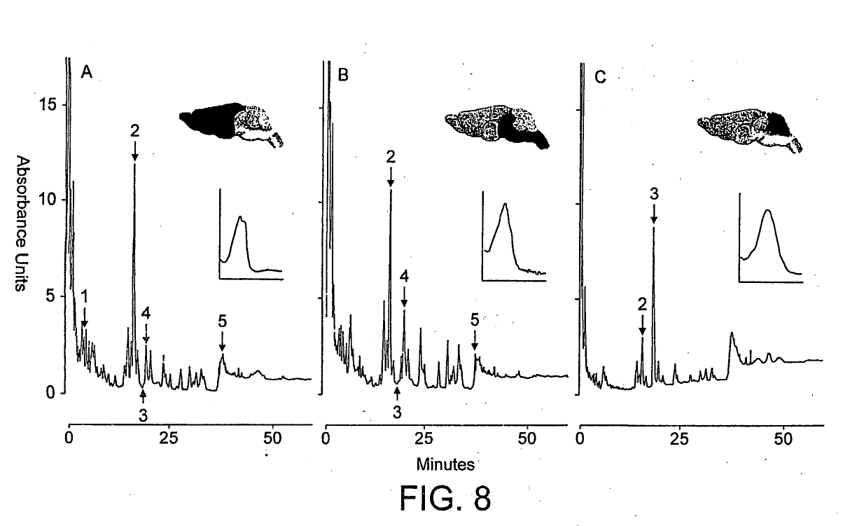

Secondly, there is an unusually large number of polar compounds which elute in the first 8 minutes (FIGS. 8A-C).

There are also characteristic features of each sample. Cerebrum (FIG. 8A) is characterised by
i) several unusual polar compounds eluting at 4-5 minutes peaks 1) which resemble 14-hydroxy-4, 14-retro-retinol (14HRR) and anyhdroretinol in having three peaks of uv absorption, although the peaks themselves are at different maxima (319 nm, 333 nm, 349 nm);
ii) a very high level of a compound eluting at 16 minutes (peak 2) which shows a 2 peak uv absorption spectrum (maxima at 309 nm and 323 nm) (inset in FIG. 8A); the absence of all-trans-retinoic acid (peak 3); the presence of a compound with a uv maximum of 322 (peak 4) which could be 4-OH-retinol (inset, FIG. 8B); very low level of all-trans-retinol (peak 5). The brainstem (FIG. 8B) is very similar except that there are no polar compounds resembling retroretinoids (peaks 1).

In contrast, the most striking features of the cerebellum samples (FIG. 8C) are the very high levels of all-trans-retinoic acid (peak 3) and the low level of the 2 peaks compound (peak 2). The uv spectrum confirms that peak 3 is all-trans-retinoic acid (inset, FIG. 8C) and its average level is determined to be 531±194 pg/mg tissue.

Thus the RARElacZ expression in the cerebellum (FIG. 7D) correlates with a high level of endogenous all-trans-RA found by HPLC.

Expression of Enzymes

The distribution of RA synthesising enzymes and components of the RA signalling machinery (binding proteins, RARs and RXRs) is examined. Based on the expression patterns disclosed herein, the present invention provides targets and/or effectors for the modulation of retinoid signalling.

The distribution of RALDH1, RALDH2, RALDH3 and CYP26 in the cerebellum of the adult mouse brain is examined. The RALDHs generate all-trans-RA from retinaldehyde and CYP26 metabolises all-trans-RA to more polar compounds such as 4-oxo-RA, 4-OH-RA, 18-OH-RA.

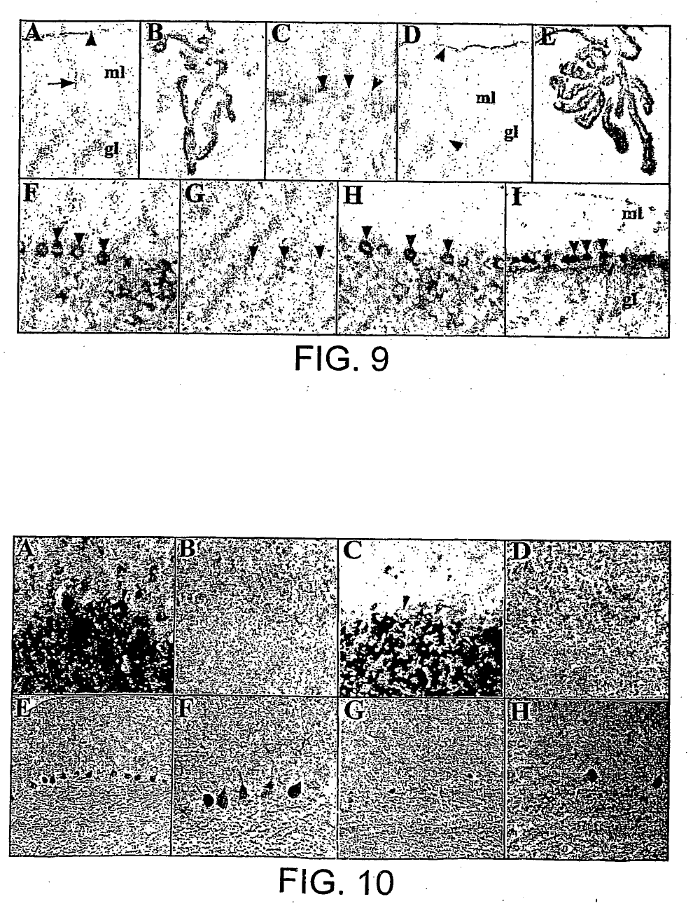

RALDH1 mRNA is not expressed in the cerebellum, only in the substantia nigral cells. RALDH2 protein is not expressed in the cerebellum itself, but in the meninges surrounding the cerebellum (FIG. 9A) and the rest of the brain. It is also expressed below the cerebellum in the choroid plexus of both the fourth ventricle and the lateral ventricle (FIG. 9B) and throughout the brain in the endothelium of the capillaries (FIG. 9A, arrow).

Without wishing to be bound by theory, RA could therefore be supplied to the neurons of the cerebellum from either of these sources, although the choroid plexus and meninges would most likely supply RA to the cerebrospinal fluid (CSF).

RALDH3 mRNA is the only RA synthesising enzyme which is expressed in the neuronal cells of the cerebellum itself, being expressed at low levels in the Purkinje cells (FIG. 9C). CYP26 mRNA, the enzyme which breaks down RA, is expressed strongly in the granule cell layer and weakly in the Purkinje cells (FIG. 10C).

Without wishing to be bound by theory, if RA is required by the neurons of the molecular layer or the granule cell layer it seems likely that it would be derived from the meninges and/or capillaries.

In contrast, the Purkinje cells express their own enzyme for RA synthesis.

Expression of Retinoid Binding Proteins

CRBP I and CRABP I are cytoplasmic proteins involved in the metabolism and sequestering of retinol and retinoic acid respectively. Disclosed herein is the extent to which these polypeptides are present in the cerebellum. Based on these disclosures, the invention provides target(s)/effector(s) for the modulation of retinol/retinoic acid signalling in adult brain.

Expression patterns are analysed using CRBP I and CRABP I antibodies.

CRBP I is present in identical locations to that of RALDH2, namely the meninges surrounding the cerebellum (FIG. 9D) and rest of the brain and the choroid plexus of the fourth and lateral ventricles (FIG. 9E).

CRABP I is expressed in relatively few cells of the choroid plexus.

Without wishing to be bound by theory, the colocalisation of CRBP I (which binds retinol and seems to be involved in its metabolism to retinal by retinol dehydrogenases (31)), and RALDH2 (which metabolises retinal to RA), strengthens the concept that the meninges and/or choroid plexus are a source of RA for the CSF. The mechanism might be summarised Meninges/Choroid plexus -> Retinol -> (action of CRBPI) -> Retinal -> (action of RALDH2) -> Retinoic acid -> CSF. Intervention at one or more point(s) in this pathway may allow modulation of RA in CSF according to the present invention.

Expression of RARs and RXRs

The expression of these nuclear transcription factors in various regions of the brain is disclosed. These observations yield information about which cells might respond to the RA that is generated. Such cells may respond for example via transcriptional activation. Thus, according to the present invention, there are provided factors capable of influencing the modulation of cellular responses to RA, which responses include transcriptional activation.

There is a differential expression of RARs and RXRs in the cerebellum. RARα is expressed strongly in the Purkinje cells and the granule cell layer (FIG. 9F) and at a low level in the choroid plexus. RARβ and RARγ are not expressed in the cerebellum (FIG. 9G). The RXRs are each expressed strongly in the Purkinje cells (FIG. 9H, I) and weakly in the granule cell layer and weakly in the choroid plexus. One difference within the RXR expression patterns is that RXRγ is additionally expressed in the molecular layer and not in the granule cell layer (FIG. 9I).

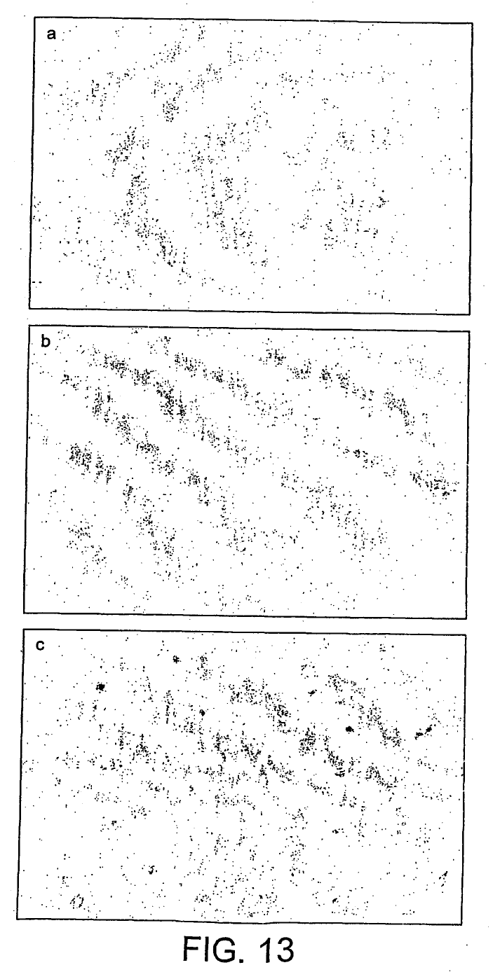

A summary diagram of these expression patterns is shown in FIG. 12.

Without wishing to be bound by theory, since RA is active in the cells of the Purkinje layer (FIG. 7D), since they express a RA synthesising enzyme and a RA metabolising enzyme and since they strongly express receptors for the activation of RA-responsive genes, it is likely that they are the key RA-dependent cells in the cerebellum, and therefore represent a key target in modulation of retinoid signalling.

Gene Expression and the Survival of the Cerebellum in the Absence of RA

Disclosed herein are the effects on Purkinje cells of the modulation of retinoid signalling. In this example, retinoid signalling is modulated via the removal of RA.

RA is removed by feeding weaned rats a vitamin A-free diet for a period of 1 year. Rats are chosen for this experiment instead of mice because mice can be more difficult to make vitamin A-deficient. Rats are the organism of choice for nutritional studies.

Using the control brains from this experiment we first confirmed that the expression data obtained in the above examples for adult mice generalises to other adult mammals, such as the adult rat cerebellum. Rats fed on a normal diet are sampled after 6 months and 1 year and the expression of the enzymes, binding proteins and receptors is studies along with an examination of the Purkinje cells themselves using a calbindin antibody. The expression of these genes is the same as in the mouse brain (cf FIGS. 9F and 10A for RARα) and the expression does not change in the control rats over a period of one year.

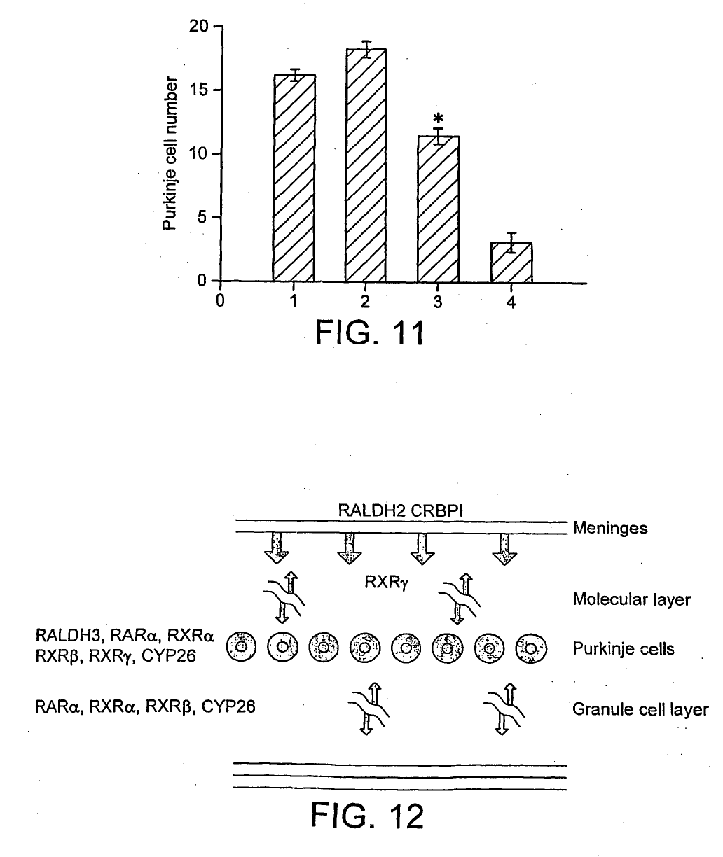

For the purposes of illustration, two gene expression patterns are shown; RARα in the Purkinje cells and granule cell layer (FIG. 10A) and CYP26 in granule cell layer and weakly in the Purkinje cells (FIG. 10C). Calbindin staining of the young adult rats, 6 month old rats and 1 year old rats does not show any differences either in staining patterns of the Purkinje cells and dendrites (FIG. 10E, F) or in the number of Purkinje cells when cell counts were performed (FIG. 11). 6 month old rats have an average of 16.3 cells per unit length (FIG. 11, column 1) and 1 year old rats have an average of 18.2 cells per unit length (FIG. 11, column 2).

After 6 months of RA deficiency there is a strong down-regulation of RARα and CYP26 in the granule cell layer and the Purkinje cells. Counts of the numbers of cells show a significant (p>0.0001) drop to 11.5 cells per unit length (FIG. 11, column 3) and there is also evidence that the dendrites are receeding. After 1 year of RA deficiency there is a complete down regulation of RARα (FIG. 10B) and CYP26 (FIG. 10D). Most dramatically one of the two animals sampled at this time point shows a massive loss of Purkinje cells in sections (FIG. 10G, H) and the cell numbers has declined to 20% of normal (FIG. 11, column 4—3.2 cells per unit length). Those remaining Purkinje cells have lost all their dendrites and are presumably non-functional (FIG. 10H). This rat showed symptoms of ataxia with a peculiar staggering gait. The other animal has a Purkinje cell number equivalent to the 6 month RA free animals along with a complete down-regulation of RARα and CYP26.

### DISCUSSION

Through study of the adult rodent brain to discover where RA and the molecular machinery involved in the transduction of the RA signal are to be found, the neuronal populations that require RA for their maintenance are determined as disclosed above.

Using a RARElacZ transgenic reporter mouse there are three regions of the adult brain which show RA activity: the hippocampus, the choroid plexus and the Purkinje cells of the cerebellum.

The role of CRBP is to interact with the retinol dehydrogenases (31) and increase the rate of synthesis of RA from RALDH2 (33). The meninges express these same two proteins (FIG. 9; (34)), and are therefore likely to synthesise RA. The high level of RA that is generated in the adult choroid plexus may be liberated into the cerebrospinal fluid (CSF).

In development it has been suggested that the choroid plexus of the fourth ventricle produces RA which is required for neurite outgrowth and morphogenesis of the cerebellum itself (32). The developing cerebellum is also sensitive to the effects of excess RA both in the human (35) and newborn rat (Yamamoto et al., 1999).

By HPLC we show that the cerebellum contains very high levels of endogenous all-trans-RA in contrast to the rest of the brain where no RA can be detected. This RA may come partly from the meninges and the choroid plexus which would have been removed with the cerebellun, but in order to determine whether there are any other intrinsic cerebellar sources of RA we examine the expression of the three RALDH enzymes which synthesise RA, namely RALDH1, 2 and 3 and CYP26, the enzyme which breaks down RA.

RALDH1 is only present in the neurons of the substantia nigra, as has been reported in embryonic and young mice (17). RALDH2 is present, as described above, in the meninges and also in the lining of the capillaries. The former location may produce RA for the CSF, but RA could also be provided to the neurons of the cerebellum from the capillaries as other nutrients are. Without wishing to be bound by theory, this would suggest that RA acts on the cerebellum in a paracrine fashion, being produced in one cell type and acting on another.

Examining RALDH3 expression reveals that the Purkinje cells themselves express a RA synthesising enzyme and this may be responsible for the RA activity that the lacZ reporter sections reveal (see above). In addition, an enzyme which breaks down RA, namely CYP26, is strongly expressed in the granule cell layer and weakly expressed in the Purkinje cells. Another CYP, P450RAI-2, is also been found to be expressed in the adult human cerebellum by Northern blot analysis (36) and thus these cells may be active in the breakdown of all-trans-RA. Surprisingly, the Purkinje cells did not express either of the cytoplasmic binding proteins, CRBP I or CRABP I.

Without wishing to be bound by theory, is possible that the Purkinje cells themselves use the RA they produce. The expression of the RARs and RXRs in the cerebellum is examined and a summary diagram of these expression patterns is shown in FIG. 12. The Purkinje cells express one RAR, RARα and each of the RXRs. It is therefore possible that the RA made by their RALDH3 is utilised in the nucleus to maintain expression of genes in the Purkinje cells themselves.

Interestingly, another receptor related to the RARs and RXRs, the orphan receptor RORα is also specifically expressed in the Purkinje cells of the adult mouse brain (38). A knockout of this gene results in a smaller cerebellum with a dramatic loss of Purkinje cells, tremor and abnormal body balance (39)). This is the same phenotype as the staggerer mutant (40) and RORα is the abnormal gene in this mouse mutant. As demonstrated herein, removal of vitamin A from the diet of rats results in a phenocopy the staggerer mutant. Without wishing to be bound by theory, it is possible that a retinoid is the ligand for RORα and in the absence of its ligand RORα is not activated, leading to lack of Purkinje cell functioning and ultimately degeneration. Thus, according to one aspect of the present invention, RORα is a candidate effector for the modulation of retinoid signalling.

Further effectors/modulators of retinoid signalling are identified by the present disclosure. For example, one gene which is known to be regulated by RA both in medulloblastoma cells (41) and chicken embryos (42) and is expressed in Purkinje cells is calbindin. This molecule is used as a marker to determine the effect on the brain of removing RA from the diet (see above). Thus, calbindin may be a further effector/modulator of retinoid signalling according to the present invention.

After 6 months of a RA free diet the experimental rats show a down-regulation of RARα and CYP26 in the Purkinje cells and granule cell layer and a significant decrease in Purkinje cell counts. After a year of a RA See diet, at least one of the two experimental animals shows clear staggering locomotor defects and has lost 80% of its Purkinje cells with the remainder showing no evidence of dendrites. These results demonstrate that retinoid signalling is a continuing requirement for the maintenance of Purkinje cells, and implicate adversely affected retinoid signalling in neurological disorders, particularly neurodegenerative disorders. Thus, modulation of retinoid signalling according to the present invention represents a valuable therapy in countering neurological disorders as described herein.

Furthermore, without wishing to be bound by theory, a mutation in one of the retinoid pathway molecules that we have shown to be expressed in Purkinje cells (RALDH3, RARα, RXRα, RXRβ, RXRγ) may be responsible for the appearance of human cerebellar ataxia. Replacing this gene or its function represents an avenue of therapy according to the present invention. Moreover, the present invention may be applied to human aging. After 6 months of a diet deficient in vitamin A our experimental rats show a decrease in expression levels of RARα and CYP26 and a decrease in Purkinje cell counts. Aging of rats is also associated with a loss of RAR and RXR mRNA in the brain which can be reversed by the administration of RA (43). The brain is also particularly affected by ageing in terms of nerve cell loss, dendritic spine reduction and loss of synaptic plasticity. Another feature of ageing is a loss of nutritional status. We can prematurely induce the neural hallmarks of ageing (decrease in RAR and RXR expression, loss of dendritic spines, loss of neural cells) by adverse modulation of retinoid signalling, such as via removal of vitamin A from the diet as described above. Therefore, the loss of nutritional status, and particularly the loss of vitamin A status, may be responsible for neural ageing. Thus, according to the present invention, a reduction in the rate of ageing process may be effected by modulation of retinoid signalling, such as by the supplementary supply of a retinoid.

### EXAMPLE 4

### Modulation of Retinoid Signalling—Addressing Alzheimer's Disease and Related Disorder(s)

Clinical properties of Alzheimer's disease are addressed, and the involvement of retinoid signalling is demonstrated.

Rats are maintained on a retinoid deficient diet as in the above examples.

Brains are sectioned and examined for the expression of beta amyloid using anti rabbit Anti-beta-amyloid 1-40, Sigma

At six months of a retinoid deficient diet there appears to be no detectable difference between the retinoid deficient rats and the normal rats. At one year of age, retinoid deficient rats show an increase in the amount of beta amyloid (FIG. 13 B and C) compared to the control (FIG. 13A). In at least one case amyloid is apparent in the blood vessels (FIG. 13 B/C) as well as the neurons.

It is demonstrated which retinoic acid receptor is deficient in the rats. At six months of age there is a decrease in the expression of RAR alpha in the cholinergic neurons of the brain of the retinoid deficient rats which is similar to the decrease in expression in neurons involved in ataxia and motoneuron disease (as discussed in the above examples).

This decrease in expression is maintained in the one year old retinoid deficient rats.

The expression of choline Acetyltransferase (CHAT), one of the enzymes involved in acetylcholine is demonstrated using anti rabbit CHAT antibody, Chemicon. The level of expression of this enzyme is decreased in the retinoid deficient rats compared to the control.

Without wishing to be bound by theory, it is disclosed that the loss of RAR alpha can lead to an increase in beta amyloid and loss of CHAT expression.

Thus, the utility of RAR alpha agonist in Alzheimer disease to prevent further production of beta amyloid is demonstrated.

### EXAMPLE 5

### Raldh-2 Gene Therapy Vector

In this example a gene therapy vector containing sequence of the Raldh-2 gene is constructed.

An oligonucleotide primer 5′ to the raldh-2 ORF and an oligonucleotide primer 3′ to the raldh-2 ORF are used to amplify mouse cDNA by PCR. The resulting PCR product encompasses the whole raldh-2 ORF. The PCR product is purified and ligated into the cloning site of a retroviral vector plasmid construct downstream of the promoter/enhancer. This construct is sequenced to verify the insert.

The construct is then transformed into a suitable retrovirus packaging cell line. Retroviral particles containing the plasmid construct are then collected.

Neuronal cells are transformed with this recombinant vector.

### EXAMPLE 6

We have further shown that in the retinoid deficient rat model (FIG. 14 lower panel) there is a loss of RARα expression in the cerebral cortex neurons, including the cholinergic neurons, compared to the normal rat (FIG. 14 top panel). Delivery of RARα according to the present invention addresses this defect.

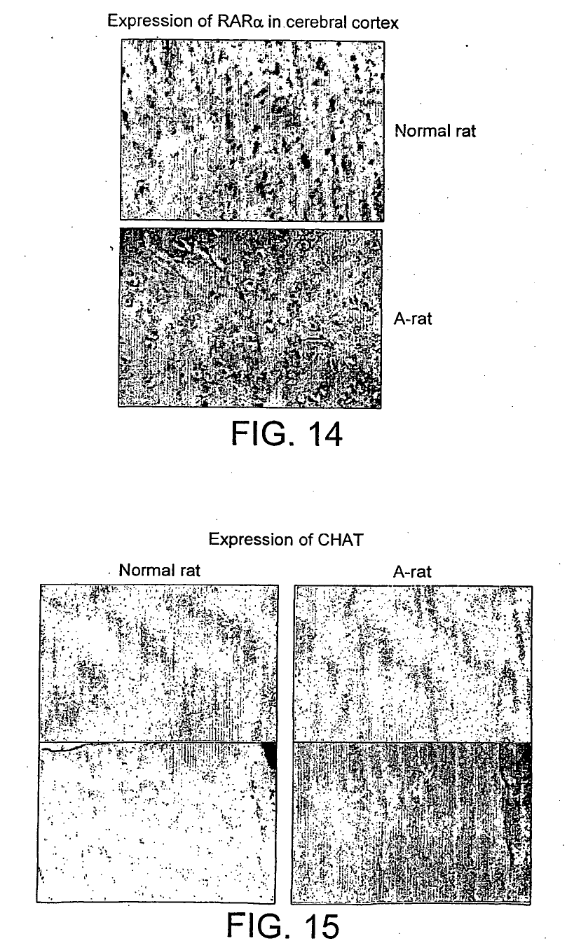

In the retinoid deficient rats, there is a loss of choline acetyl transferase (CHAT) in the cholinergic neurons of the cerebral cortex (FIG. 15 right panel) compared to normal fed rats (FIG. 15 left panel). CHAT is involved in the production of the neurotransmitter acetylcholine, which is lost in Alzheimer's diseased patients. In our rat model of neurodegenerative disease, CHAT precedes the deposition of β amyloid. Thus it is demonstrated-that the modulation of β amyloid production is effected via the upstream elements RARα and acetylcholine. RARα regulates the production of acetylcholine, which may then regulate β amyloid production. Furthermore, RARα may regulate the production of other neurotransmitters involved in Alzheimer's disease. Thus, by delivering RARα according to the present invention, defects involving β amyloid production may be addressed.

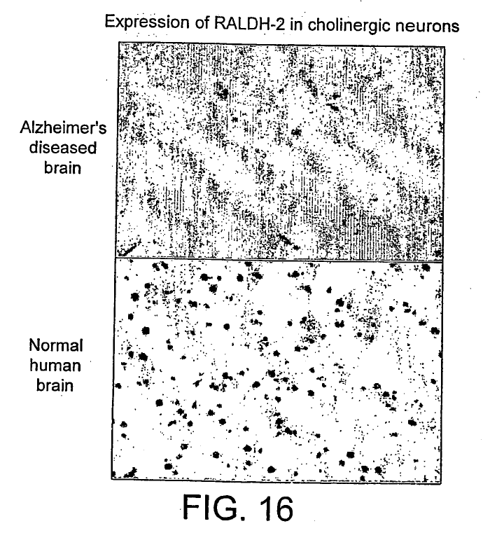

In humans, the cerebral cortex neurons (unlike the rat cerebral cortex neurons) express the retinoic acid synthesising enzyme Raldh-2. We demonstrate that this Raldh-2 enzyme is dramatically down regulated in Alzheimer's diseased patients (FIG. 16 top panel) compared to non diseased patients (FIG. 16 lower panel). Thus it is disclosed that human cerebral cortex neurons have an extra source of RA in that they are able to synthesise it by expression of Raldh-2. This extra source is in addition to the production of RA by the meninges. Thus it is disclosed that the loss of expression of this Raldh-2 enzyme leads to β amyloid deposition. Provision of Raldh-2 according to the present invention addresses this defect.

### EXAMPLE 7

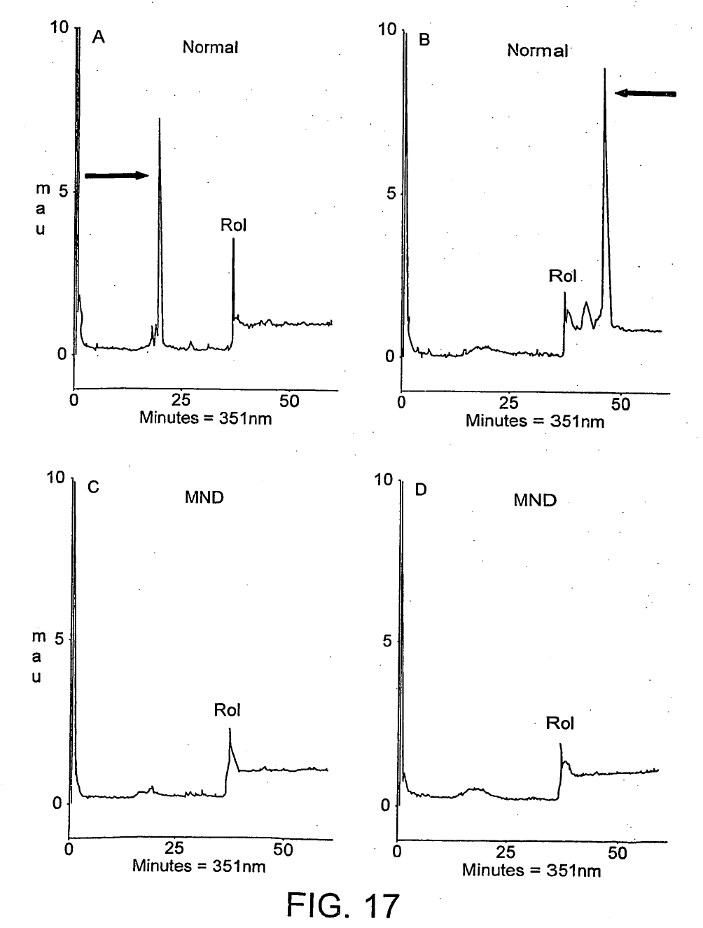

We have further shown in samples from human subjects with motoneuron disease analysed by HPLC that there is a loss of novel retinoic acids in these samples compared to the non diseased samples (FIG. 17).

The identification of these retinoic acids is important for the design of retinoids for the treatment of motoneuron disease. Thus, the present invention relates to such candidate retinoic acids for the treatment of motoneuron disease.

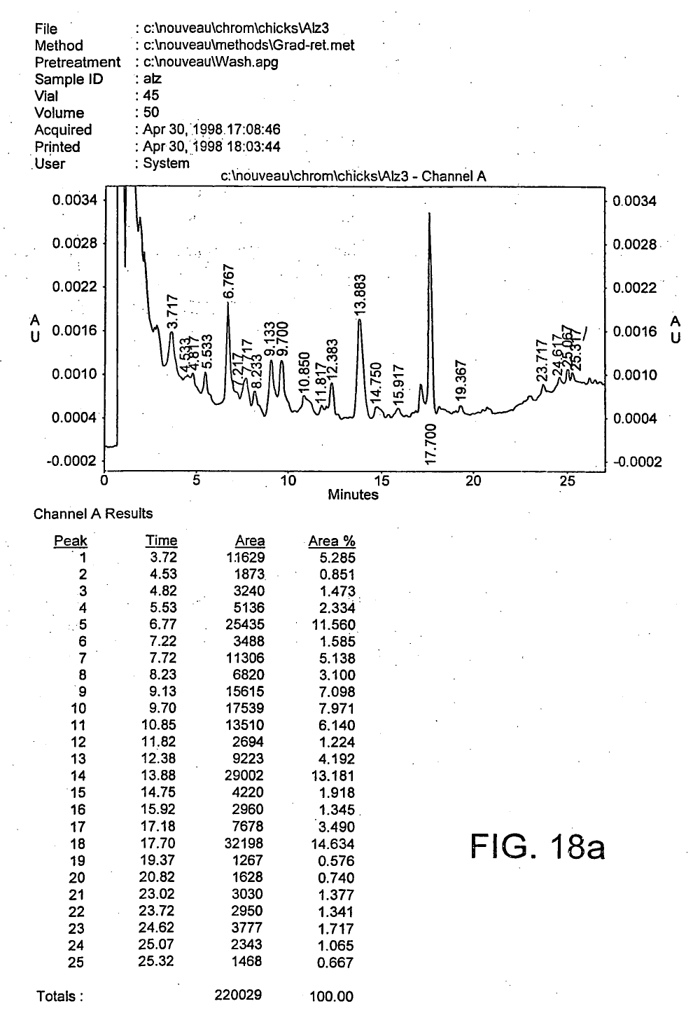

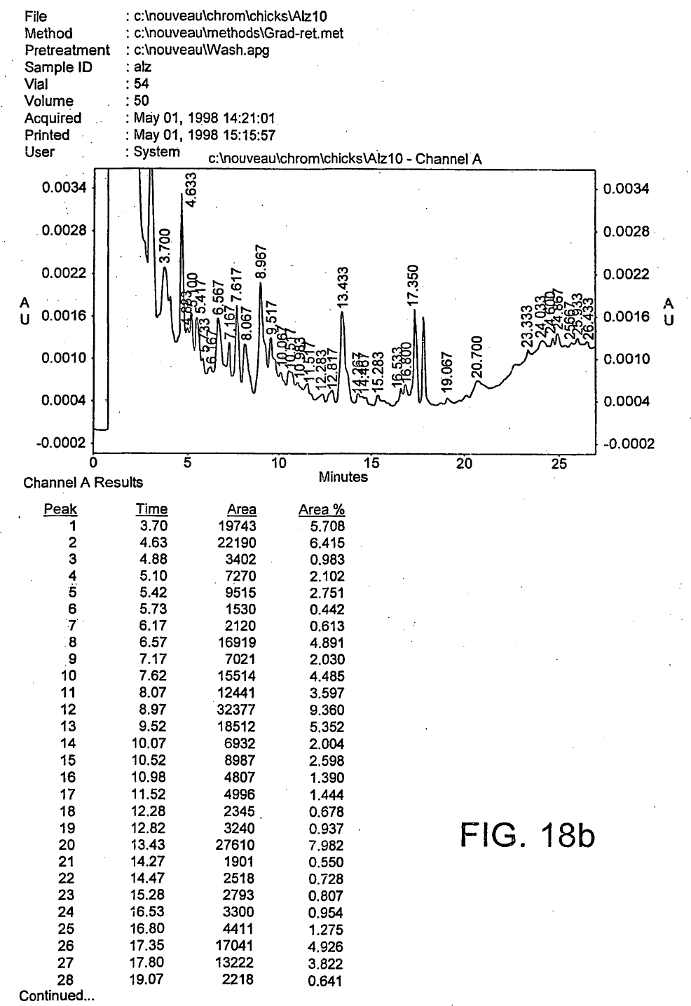

Similarly, we disclose that in human Alzheimer diseased and normal cerebral cortex there are a number of novel retinoids. Strikingly, we demonstrate that in the Alzheimer diseased brains there is a loss of retinoic acid compared to the non diseased brains (FIG. 18). Thus, it can be seen that provision of retinoic acid according to the present invention addresses problems of Alzheimer's disease.

### REFERENCES

Corcoran, J. & Maden, M (1999). Nat Neurosci. 2,307-308.
Corcoran, J., et al (2000). J. Cell Sci. 113, 2567-2574.
Delisle, M. B. & Carpenter, S (1984) J. Neurol. Sci. 63, 241-250.
Deng, H. X. et al. (1993) Science 261, 1047-1051.
Duester G. (2000) Eur J Biochem 267: 4315-4324.
Jackson, C. E. & Bryan, W. W. (1998) Semin. Neurol. 18,27-39.
Kastner, P., Chambon, P. & Leid, M (1994) In Vitamin A in Health and Disease. (R Blomhoff ed. ) pp 189-238. Marcel Dekker Inc., New York.
Kastner P et al. (1995) Cell 83:859-869.
Kliewer, S. A., et al (1994) In Vitamin A in Health and Disease. (R. Blomhoff ed.) pp 239-255. Marcel Dekker Inc., New York.
Lehmann J M et al. (1992) Science 258: 1944-1946.
Maden, M. (1998). Biochem. 30, 81-111.
Mangelsdorf, D. J. & Evans, R. M. (1995) Cell 83, 841-850.
Mulder, D. W., Kurland, L. T., Offord, K. P. & Beard, C. M (1986) Neurology 36, 511-517.
Nagpal S. and Chandraratna R A S. (1998) Curr Opin Clin Nutr Metab Care 1: 341-346.
Nagpal S. et al. (1992) Cell 70:1007-1019.
Quinn, S. D. & De Boni, U. (1991) In Vitro Cell Dev. Biol. 27, 55-62.
Rosen, D. R. (1993) Nature 364, 362.
Ross S A et al. (2000). Physiological Reviews 80:1021-1054.
Wald G. (1951) Science 113; 287-291.
Wuarin, L. & Sidell, N. (1991). Dev. Biol. 144, 429-435.

### REFERENCES TO EXAMPLE 3

1. Kastner, P., Chambon, P. & Leid, M. (1994) in Vitamin A in Health and Disease, ed. Blomhoff, R. (Marcel Dekker Inc. New York), pp. 189-238.

2. Kliewer, S. A., Umesono, K., Evans, R. M. & Mangelsdorf, D. J. (1994) in Vitamin A in Health and Disease, ed. Blomhoff, R. (Marcel Dekker Inc., New York), pp. 239-255.

3. Duester, G. (1996) Biochemistry 35, 12221-12227.

4. Fujii, H., Sato, T., Kaneko, S., Gotoh, O., Fujii-Kuriyama, Y., Osawa, K., Kato, S. & Hamada, H. (1997) EMBO J. 16, 4163-4173.

5. White, J. A., Guo, Y.-D., Baetz, K., Beckett-Jones, B., Bonasoro, J., Hsu, K. E., Dilworth, F. J., Jones, G. & Petkovich, M. (1996) J. Biol. Chem. 271, 29922-29927.

6. Maden, M. Int. Rev. Cytol 2001 (in press).

7. Malpel, S., Mendelsohn, C. & Cardoso, W. V Development 127, 3057-3067. 2000.

8. Lu, H.-C., Reveili, J.-P., Goering, L., Thaller, C. & Eichele, G. (1997) Development 124, 1643-1651.

9. Stratford, T., Horton, C. & Maden, M. (1996) Current Biology 6, 1124-1133.

10. Mendelsohn, C., Batourina, E., Fung, S., Gilbert, T. & Dodd, J. (1999) Development 126, 1139-1148.

11. Maden, M., Gale, E., Kostetskii, I. & Zile, M. (1996) Current Biol. 6, 417-426.

12. White, J. C., Highland, M., Kaiser, M. & Clagett-Dame, M. Dev. Biol. 220, 263-284. 2000.

13. Wolbach, S. B. & Howe, P. R. (1925) J. Exp. Med 42, 753-777.

14. Krezel, W., Kastner, P. & Chambon, P. Neuroscience 89, 1291-1300. 1999.

15. Macdonald, P. N., Bok, D. & Ong, D. E. P.N.A.S. USA 87, 4265-4269. 1990.

16. Zetterstrom, R. H., Simon, A., Giacobini, M. M. J., Eriksson, U. & Olson, L. (1994) Neuroscience 62, 899-918.

17. McCaffery, P. & Drager, U. C. (1994) Proc. Natl. Acad Sci. USA 91, 7772-7776.

18. Krezel, W., Ghyselinck, N., Samad, T. A., Dupe, V., Kastner, P., Borelli, E. & Chambon, P. (1998) Science 279, 863-867.

19. Chiang, M.-Y., Misner, D., Kempermann, G., Schikorski, T., Giguere, V., Sucov, H. M., Gage, F. H., Stevens, C. F. & Evans, R. M. (1998) Neuron 21, 1353-1361.

20. Mendelsohn, C., Ruberte, E., LeMeur, M., Morriss-Kay, G. & Chambon, P. (1991) Development 113, 723-734.

21. Thaller, C. & Eichele, G. (1987) Nature 327, 625-628.

22. Achkar, C. C., Derguini, F., Blumberg, B., Langston, A., Levin, A. A., Speck, J., Evans, R. M., Boldano, J., Nakanishi, K., Buck, J. et al. (1996) Proc. Natl. Acad. Sci. USA 93, 4879-4884.

23. Eriksson, U., Das, K., Busch, C., Norlinder, H., Rask, L., Sundellin, J., Sallstrom, J. & Peterson, P. A. (1984) J. Biol. Chem. 259, 13464-13470.

24. Eriksson, U., Hansson, E., Norlinder, H., Busch, C., Sundelin, J. & Peterson, P. A. (1987) J. Cell Physiol. 133, 482-490.

25. Berggren, K., McCaffery, P., Drager, U. & Forehand, C. J. (1999) Dev. Biol. 210, 288-304.

26. Corcoran, J., Shroot, B., Pizzey, J. & Maden, M. (2000) J. Cell Sci. 113, 2567-2574.

27. Dolle, P., Ruberte, E., Leroy, P., Morriss-Kay, G. & Chambon, P. (1990) Development 110, 1133-1151.

28. Dolle, P., Fraulob, V., Kastner, P. & Chambon, P. (1994) Mech. of Dev. 45, 91-104.

29. Swindell, E. C., Thaller, C., Sockanathan, S., Petkovich, M., Jessell, T. M. & Eichele, G. (1999) Dev. Bio. 216, 282-296.

Hind, M., Corcoran, J. & Maden, M. (2001) Am. J. Resp. Cell Mol Biol.

31. Napoli, J. L. (1994) in Vitamin A in Health and Disease, ed. Blomhoff, R. (Marcel Dekker Inc., New York), pp. 135-188.

32. Yamamoto, M., McCaffery, P. & Drager, U. C. (1996) Dev. Brain Res. 93, 182-190.

33. Yamamoto, M., Drager, U. C., Ong, D. E. & McCaffery, P. (1998) Eur. J. Biochem. 257, 344-350.

34. McCaffery, P. & Drager, U. C. (1995) in Enzymology and Molecular Biology of Carbonyl Metabolism., ed. al., H. W. e. (Plenum Press, New York), pp. 173-183.

35. Lammer, E. J. & Armstrong, D. L. (1992) in Retinoids in Normal and Development and Teratogenesis, ed. Morriss-Kay, G. (Oxford University Press, Oxford), pp. 281-295.

36. White, J. A., Ramshaw, H., Taimi, M., Stangle, W., Zhang, A., Everingbam, S., Creighton, S., Tam, S.-P., Jones, G. & Petkovich, M. (2000) Proc. Natl. Acad Sci. USA 97, 6403-6408.

37. Zetterstrom, R. H., Lindqvist, E., Mata de Urquiza, A., Tomac, A., Eriksson, U., Perlmann, T. & Olson, L. (1999) Eur. J. Neurosci. 11, 407-416.

38. Matsui, T., Sashihara, S., Oh, Y. & Waxman, S. G. Mol. Brain. Res. 33, 217-226. 1995.

39. Dussault, I., Fawcett, D., Matthyssen, A., Bader, J.-A. & Giguere, V. Mech. Dev. 70, 147-153. 1998.

40. Hamilton, B. A., Frankel, W. N., Kerrebrock, A. W., Hawkins, T. L., FitzHugh, W., Kusumi, K., Russell, L. B., Mueller, K. L., van Berkel, V., Birren, B. W. et al. (1996) Nature 379, 736-739.

41. Wang, Y.-Z. & Christakos, S. (1995) Mol. Endocrinol. 9, 1510-1521.

42. Matsumoto, K., Ieda, T., Saito, N., Ono, T. & Shimada, K. Comp. Biochem. Physiol. A 120, 237-242. 1998.

Enderlin, V., Pallet, V., Alfos, S., Dargelos, E., Jaffard, R., Garcin, H. & Higueret, P. Neurosci. Lett. 229, 125-129. 1997.

1. A method for treating a condition in a subject comprising administering an effective amount of an agent to said subject wherein said agent modulates one or more component(s) of the retinoid signalling pathway.

2. A method according to claim 1 wherein said condition is a neurological condition such as a motor neurone disease, a cerebral dementing disorder or a degenerative movement disorder.

3. A method according to claim 1 wherein said component of the retinoid signalling pathway is an aldehyde dehydrogenase.

4. A method according to claim 3 wherein said aldehyde dehydrogenase is retinaldehyde dehydrogenase 2 (RALDH-2).

5. A method according to claim 1 or claim 2 wherein said component of the retinoid signalling pathway is a retinoid receptor.

6. A method according to claim 5 wherein said retinoid receptor is retinoic acid receptor α.

7. A method for treating a condition in a subject comprising administering an effective amount of an agent to a subject wherein said agent -modulates the expression of one or more component(s) of the retinoid signalling pathway.

8. A method according to claim 7 wherein said condition is a neurological condition such as a motor neurone disease, a cerebral dementing disorder or a degenerative movement disorder.

9. A method according to claim 7 or claim 8 wherein said component is a gene encoding an aldehyde dehydrogenase.

10. A method according to claim 9 wherein said aldehyde dehydrogenase gene is a retinaldehyde dehydrogenase 2 (raldh-2).

11. A method according to any preceding claim wherein said agent comprises raldh-2.

12. A method according to claim 7 wherein said component is a gene encoding a retinoid receptor.

13. A method according to claim 12 wherein said retinoid receptor gene encodes retinoic acid receptor α.

14. A method according to any of claims 1-10, 12 or 13 wherein said agent comprises a retinoid receptor gene.

15. A method according to claim 7 wherein said component is a gene encoding a retinoic acid responsive gene.

16. A method according to claim 15 wherein said retinoic acid responsive gene encodes Islet-1.

17. A method according to any of claims 1-10, 12, 13, 15 or 16 wherein said agent comprises a retinoic acid responsive gene.

18. A pharmaceutical composition comprising a RALDH-2 polypeptide, or a fragment, variant or derivative thereof, or a polynucleotide encoding same, and a pharmaceutically acceptable carrier, diluent or excipient therefor.

19. Use of a RALDH-2 polypeptide, or a fragment, variant or derivative thereof, or a polynucleotide encoding same, in the manufacture of a medicament for treatment of a neurological condition.

20. A gene therapy vector comprising a retinoid receptor gene or a fragment, variant or derivative thereof.

21. A gene therapy vector according to claim 20 wherein said retinoid receptor gene encodes retinoic acid receptor α.

22. A gene therapy vector comprising an aldehyde dehydrogenase gene or a fragment, variant or derivative thereof.

23. A gene therapy vector according to claim 22 wherein said aldehyde dehydrogenase gene encodes RALDH-2.

24. A gene therapy vector comprising a retinoic acid responsive gene or a fragment, variant or derivative thereof.

25. A gene therapy vector according to claim 24 wherein said retinoic acid responsive gene encodes Islet-1.

26. A gene therapy vector comprising the mouse or human raldh-2 gene or a fragment, variant or derivative thereof.
